(19) United States
(12) Patent Application Publication
(10) Pub. No.: US 2012/0277316 A1
(43) Pub. Date: Nov. 1, 2012

(54) METHODS AND COMPOSITIONS FOR TREATING AND PREVENTING PARENTERAL NUTRITION ASSOCIATED LIVER DISEASE

(75) Inventors: Emma Tillman, Memphis, TN (US); Richard A. Helms, Memphis, TN (US); Michael Storm, Germantown, TN (US)

(73) Assignee: University of Tennesse Research Foundation a university

(21) Appl. No.: 13/520,079

(22) PCT Filed: Jan. 19, 2011
(86) PCT No.: PCT/US2011/021691

§ 371 Date: Jun. 29, 2012

Related U.S. Application Data
(60) Provisional application No. 61/296,243, filed on Jan. 19, 2010.

(51) Int. Cl. A61K 31/202; A61P 1/16; A61K 31/232

(52) U.S. Cl. 514/547; 514/560; 514/549

(57) ABSTRACT

Methods and compositions for treating or preventing parenteral nutrition associated liver disease are provided. Methods and compositions for advancing enteral tolerance in subjects receiving enteral nutrition are provided. The methods involve the use of omega-3 fatty acid compositions. In some embodiments the omega-3 fatty acid compositions comprise docosahexanoic acid and eicosapentaenoic acid. In some embodiments the omega-3 fatty acid compositions comprise fish oil. In some embodiments the subjects to be treated are receiving parenteral nutrition. In some embodiments the subjects to be treated are infants having a low birth weight, very low birth weight, extremely low birth weight, a low gestational age, short bowel syndrome, necrotizing entercolitis, or any combination thereof.

### CROSS REFERENCE TO RELATED APPLICATIONS

This application claims the benefit of U.S. Provisional Patent Application Ser. No. 61/296,243, filed Jan. 19, 2010, the disclosure of which is incorporated herein by reference in its entirety.

### TECHNICAL FIELD

The presently disclosed subject matter generally relates to methods and compositions for treating or preventing parenteral nutrition associated liver disease. More particularly, the presently disclosed subject matter provides methods and compositions, including omega-3 fatty acid compositions in some embodiments, for advancing enteral tolerance in subjects receiving enteral nutrition. In some embodiments, the presently disclosed methods and compositions are used to treat infants.

### ABBREVIATIONS

PN parenteral nutrition

TPN total parenteral nutrition

PNALD parenteral nutrition associated liver disease

IVFE intravenous fat emulsions

EN enteral nutrition

EPA eicosapentaenoic acid

DHA docosahexaenoic acid

SBS short bowel syndrome

NG nasogastric

G gastrostomy

NEC necrotizing enterocolitis

NPO nil per os (nothing by mouth)

INR international normalized ratio

ROS reactive oxygen species

PPAR-α peroxisome proliferator-activated receptor alpha

NF-κB nuclear factor-kappaB

USP United States Pharmacopeia

AST aspartate aminotransferase

ALT alanine aminotransferase

LBW low birth weight

VLBW very low birth weight

ELBW extremely low birth weight

GGT gamma-glutamyl transpeptidase

Alk Phos alkaline phosphatase

CDCA chenodeoxycholic acid

### BACKGROUND

Nutrition support through parenteral nutrition (PN) is necessary when patients cannot be fed orally, for example when a patient has an impaired gastrointestinal tract and is unable to tolerate enteral feedings. Parenteral nutrition associated liver disease (PNALD) occurs in approximately 25-66% of infants and children maintained on long term PN and the incidence increases in infants with low birth weight, very low birth weight, extremely low birth weight, low gestational age and those with short bowel syndrome (SBS) (Merritt, 1986; Beale et al., 1979). Hepatocellular injury can be observed as early as two to four weeks after initiation of PN (Merritt, 1986). If not reversed, PNALD can progress from cholestasis to liver fibrosis, hepatic failure and death (Beale et al., 1979). Even prior to the use of PN, an increase in bile stasis was observed in neonates with intestinal anomalies and sepsis that precluded enteral nutrition (EN) (Nakai & Landing, 1961).

While most PNALD reverses with the discontinuation of PN and the initiation of EN, many patients with SBS are dependent on PN support. Thus, there remains a need for an effective treatment for PNALD.

### BRIEF DESCRIPTION OF THE DRAWINGS

FIG. 1 is a graph showing bilirubin in relation to enteral feeding for patient 1. Baseline and follow-up values for total bilirubin (solid line with solid diamonds) are plotted in comparison to percentage enteral intake (dashed line with gray squares). Initiation of enteral fish oil therapy (0.15 g/kg/day) is denoted by the solid arrow at time point A.

FIG. 2 is a graph showing bilirubin in relation to enteral feeding for patient 2. Baseline and follow-up values for total bilirubin (solid line with solid diamonds) are plotted in comparison to percentage enteral intake (dashed line with gray squares). Initiation of enteral fish oil therapy (0.87 g/kg/day) is denoted by the solid arrow at time point A.

FIG. 3 is a graph showing bilirubin in relation to enteral feeding for patient 3. Baseline and follow-up values for total bilirubin (solid line with solid diamonds) are plotted in comparison to percentage enteral intake (dashed line with gray squares). Initiation of enteral fish oil therapy (1 g/kg/day) is denoted by the solid arrow at time point A.

FIG. 4 is a graph showing bilirubin in relation to enteral feeding for patient 4. Baseline and follow-up values for total bilirubin (solid line with solid diamonds) are plotted in comparison to percentage enteral intake (dashed line with gray squares). Initiation (0.8 g/kg/day, time point A) and subsequent discontinuation (time point B) of enteral fish oil therapy is denoted by the solid arrows.

FIG. 5 is a graph showing bilirubin in relation to enteral feeding for patient 5. Baseline and follow-up values for total bilirubin (solid line with solid diamonds) are plotted in comparison to percentage enteral intake (dashed line with gray squares). Initiation of enteral fish oil therapy (0.6 g/kg/day) is denoted by the solid arrow at time point A.

FIG. 6 is a graph showing bilirubin in relation to enteral feeding for patient 6. Baseline and follow-up values for total bilirubin (solid line with solid diamonds) are plotted in comparison to percentage enteral intake (dashed line with gray squares). Initiation (0.6 g/kg/day, time point A) and temporary withholding (time point B) of enteral fish oil therapy is denoted by the solid arrows.

FIG. 7 is a bar graph showing the effects of EPA and DHA, alone or in combination, on caspase 3/7 activity in HepG2 cells exposed to chenodeoxycholic acid (CDCA). Caspase 3/7 activity was determined by measuring fluorescence, expressed as relative fluorescence units (RFU), in cells incubated under the following treatment conditions: vehicle EtOH alone (control), CDCA, EPA, DHA, EPA+DHA, CDCA+EPA, CDCD+DHA, and CDCA+EPA+DHA.

FIG. 8 is a bar graph showing the effects of EPA on cell viability in HepG2 cells exposed to chenodeoxycholic acid (CDCA). Cell viability is demonstrated by trypan blue staining. Viable cells are represented in the dark shaded bars and dead cells are represented by the light bars.

FIGS. 9, 10 and 11 are bar graphs showing the effects of EPA and DHA on caspase 3/7 activity in HepG2 cells exposed to chenodeoxycholic acid (CDCA). FIG. 9 shows a time course with CDCA 200 μM±EPA 10 μM. FIG. 10 shows a dose response shown at 12 hours. FIG. 11 shows synergy of omega-3 fatty acids (EPA and DHA) at 12 hours. Data are represented based on relative fluorescence units above control. Each bar represents mean±SEM for data from three independent experiments. Statistical significance was determined using an ANOVA with Tukey's LSD. Treatment conditions represented with different symbols above the bars are significantly different at p<0.05. Those with the same or no symbols are not significantly different.

FIGS. 12 and 13 are bar graphs showing the effects of EPA and DHA on Fas (FIG. 12) and TRAIL-R2 (FIG. 13) mRNA expression levels. Fas and TRAIL-R2 mRNA levels were measured by quantitative RT-PCR. Values are based on a fold change relative to the vehicle control. Statistical significance was determined using an ANOVA with Tukey's LSD. Each bar represents mean±SEM for data from three culture wells. Those with the same or no symbols are not significantly different.

FIG. 14 is a bar graph showing the effects of EPA on pro-inflammatory cytokine (IL-6) mRNA expression in HepG2 cells exposed to chenodeoxycholic acid (CDCA). IL6 mRNA levels were measured by quantitative RT-PCR after a 2 hour incubation in HepG2 cells. Values are based on a fold change relative to the vehicle control. Each bar represents mean±SEM for data from three culture wells. Statistical significance was determined using an ANOVA with Tukey's LSD. Treatment conditions represented with different symbols above the bars are significantly different at p<0.05. Those with the same or no symbols are not significantly different.

### SUMMARY

In some embodiments the presently disclosed subject matter comprises a method of treating parenteral nutrition associated liver disease (PNALD) in a subject, the method comprising: providing a subject with PNALD; and administering to the subject an effective amount of an omega-3 fatty acid composition, wherein the omega-3 fatty acid composition is administered enterally. In some embodiments, the subject is an infant having a low birth weight, very low birth weight, extremely low birth weight, a low gestational age, short bowel syndrome (SBS), necrotizing entercolitis (NEC), gastroeschesis, omphelacele, atresias, Hirschprungs disease, functional short bowel syndrome or a combination thereof. In some embodiments, a subject with PNALD is a subject having a direct bilirubin concentration of >2 mg/dL and/or elevated transaminases, GGT, alk phos, or clinical correlation. In some embodiments, the omega-3 fatty acid composition comprises docosahexanoic acid (DHA) and eicosapentaenoic acid (EPA). In some embodiments, the omega-3 fatty acid composition comprises fish oil or deodorized fish oil. In some embodiments, the omega-3 fatty acid composition comprises algal sourced omega-3 fatty acids. In some embodiments, the algal sourced omega-3 fatty acids are in the triglyceride or ethyl ester form. In some embodiments, the subject is receiving parenteral nutrition (PN). In some embodiments, the enteral administration comprises oral administration. In some embodiments, treatment comprises an enhanced ability by the subject to tolerate enteral feeding as compared to a subject receiving PN but not administered an effective amount of an omega-3 fatty acid composition.

In some embodiments, the presently disclosed subject matter provides a method of preventing PNALD in a subject, the method comprising: providing a subject at risk for developing PNALD; and administering to the subject an effective amount of an omega-3 fatty acid composition, wherein the omega-3 fatty acid composition is administered enterally. In some embodiments, the subject at risk for developing PNALD is an infant receiving PN and having a low birth weight, very low birth weight, extremely low birth weight, a low gestational age, SBS, NEC, gastroeschesis, omphelacele, atresias, Hirschprungs disease, functional short bowel syndrome or a combination thereof. In some embodiments, the omega-3 fatty acid composition comprises DHA and EPA. In some embodiments, the omega-3 fatty, acid composition comprises fish oil or deodorized fish oil. In some embodiments, the omega-3 fatty acid composition comprises algal sourced omega-3 fatty acids. In some embodiments, the algal sourced omega-3 fatty acids are in the triglyceride or ethyl ester form. In some embodiments, the enteral administration comprises oral administration. In some embodiments, the enteral administration of the omega-3 fatty acid composition is concurrent with PN. In some embodiments, preventing comprises an enhanced ability for the subject to tolerate enteral feeding as compared to a subject receiving PN but not administered an effective amount of an omega-3 fatty acid composition.

In some embodiments the presently disclosed subject matter provides a method of advancing enteral tolerance in a subject receiving parenteral nutrition, the method comprising: providing a subject receiving parenteral nutrition; and administering to the subject an effective amount of an omega-3 fatty acid composition, wherein the omega-3 fatty acid composition is administered enterally. In some embodiments, the subject is an infant having a low birth weight, very low birth weight, extremely low birth weight, a low gestational age, SBS, NEC, gastroeschesis, omphelacele, atresias, Hirschprungs disease, functional short bowel syndrome or a combination thereof. In some embodiments, the subject is diagnosed with PNALD, wherein PNALD is defined as having a direct bilirubin concentration of >2 mg/dL and/or elevated transaminases, GGT, alk phos, or clinical correlation. In some embodiments, the omega-3 fatty acid composition comprises DHA and EPA. In some embodiments, the omega-3 fatty acid composition comprises fish oil or deodorized fish oil. In some embodiments, the omega-3 fatty acid composition comprises algal sourced omega-3 fatty acids. In some embodiments, the algal sourced omega-3 fatty acids are in the triglyceride or ethyl ester form. In some embodiments, the enteral administration comprises oral administration. In some embodiments, the enteral administration of the omega-3 fatty acid composition is concurrent with PN. In some embodiments, advancing enteral tolerance comprises an enhanced ability for the subject to tolerate enteral feeding as compared to a subject receiving PN but not administered an effective amount of an omega-3 fatty acid composition.

In some embodiments, the presently disclosed subject matter provides a palatable omega-3 fatty acid composition comprising DHA and EPA for enteral administration. In some embodiments, the omega-3 fatty acid composition comprises fish oil. In some embodiments, the fish oil comprises deodorized fish oil. In some embodiments, the composition further comprises a flavoring and/or masking agent. In some embodiments, the omega-3 fatty acid composition comprises algal sourced omega-3 fatty acids as triglyceride or ethyl ester. In some embodiments, the algal sourced omega-3 fatty acids are in the triglyceride or ethyl ester form. In some embodiments, the composition comprises a DHA:EPA ratio of 1:3 to 3:1 In some embodiments, the composition is in an oil-in-water emulsion or a powder-in-liquid suspension. In some embodiments, the composition comprises a 50:50 (vol/vol) oil-in-water emulsion, wherein the oil is a fish oil mixture of EPA and DHA. In some embodiments, the composition comprises a 50:50 (vol/vol) oil-in-water emulsion, wherein the oil is a steam de-odorized fish oil mixture of EPA and DHA. In some embodiments, the composition comprises a 40:60 (vol/vol) oil-in-water emulsion, wherein the oil is an algal oil mixture of EPA and DHA. In some embodiments, the algal oil mixture is in the triglyceride or ethyl ester form.

In some embodiments, the presently disclosed subject matter provides a method of treating or preventing an inflammatory disease in a subject, the method comprising: providing a subject having or at risk for developing a disease with an inflammatory component; and administering to the subject an effective amount of an omega-3 fatty acid composition. In some embodiments, the inflammatory disease comprises pediatric or adult inflammatory bowel disease, cystic fibrosis, critical illness, burns, metabolic syndrome, obesity, malignancy related weight loss, bipolar disorder, cardiovascular disease or a combination thereof.

In some embodiments, the presently disclosed subject matter provides a method of attenuating hepatocellular apoptosis in a subject receiving parenteral nutrition or suffering from PNALD, the method comprising: providing a subject receiving parenteral nutrition or a subject suffering from PNALD; and administering to the subject an effective amount of an omega-3 fatty acid composition, wherein the omega-3 fatty acid composition is administered enterally. In some embodiments, the subject has levels of retained hydrophilic bile salts that are higher than the levels of retained hydrophilic bile salts in a subject not receiving parenteral nutrition or suffering from PNALD. the subject is an infant having a low birth weight, very low birth weight, extremely low birth weight, a low gestational age, short bowel syndrome (SBS), necrotizing entercolitis (NEC), gastroeschesis, omphelacele, atresias, Hirschprungs disease, functional short bowel syndrome or a combination thereof. In some embodiments, a subject with PNALD is a subject having a direct bilirubin concentration of >2 mg/dL and/or elevated transaminases, GGT, alk phos, or clinical correlation. In some embodiments, the omega-3 fatty acid composition comprises docosahexanoic acid (DHA) and eicosapentaenoic acid (EPA). In some embodiments, the omega-3 fatty acid composition comprises fish oil or deodorized fish oil. In some embodiments, the omega-3 fatty acid composition comprises algal sourced DHA and EPA. In some embodiments, the enteral administration comprises oral administration. In some embodiments, attenuating hepatocellular apoptosis comprises reducing the level of hepatocellular apoptosis to a level that is lower than the level of hepatocellular apoptosis in a subject receiving parenteral nutrition or suffering from PNALD but not receiving an effective amount of an omega-3 fatty acid composition.

It is an object of the presently disclosed subject matter to provide methods and compositions for treating and preventing parenteral nutrition associated liver disease. This and others objects are achieved in whole or in part by the presently disclosed subject matter.

An object of the presently disclosed subject matter having been stated above, other objects and advantages of the presently disclosed subject matter will become apparent to those skilled in the art after a study of the following description, Figures and Examples.

### DETAILED DESCRIPTION

I. Definitions

While the following terms are believed to be well understood by one of ordinary skill in the art, the following definitions are set forth to facilitate explanation of the presently disclosed subject matter. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the articles “a”, “an”, and “the” refer to “one or more” when used in this application, including in the claims. For example, the phrase “a marker” refers to one or more markers. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the terms “effective amount” and “therapeutically effective amount” are used interchangeably and mean a dosage sufficient to provide treatment for the disease state being treated. This can vary depending on the patient, the disease and the treatment being effected.

As used herein “parenteral nutrition”, “PN”, “total parenteral nutrition” or “TPN” refers to a regimen of obtaining nutrition by a parenteral route when enteral (oral or gastrointestinal) nutrition is impossible or impaired. Such conditions may occur in certain disease states and/or in newborn infants. PN is generally administered to the patient via an intravenous route, either in a central or peripheral vein. Any other known route of administering PN is also within the scope of the presently disclosed subject matter, for example but not limited to, intraperitoneal. PN solutions are usually administered continuously by intravenous infusion, but can be delivered intermittently in some embodiments. The dosage of nutrients administered during PN is determined by the total body weight, estimated requirements, and status of the patient. The dosage is then typically expressed as the dosage of nutrients/kg body weight/24 h period. One skilled in the art can readily determine the proper dosage and rate of administration to achieve the desired nutritional state. The optimal mixture of nutrients is one which will produce a normal pattern of metabolites and nutritive components as well as appropriate growth for infants and children.

The nutritive requirements for PN are well known, PN solutions having first been developed in the 1950s. These solutions must provide all nutrients including an energy source (e.g. carbohydrates), amino acids (as a substitute for protein), lipids, vitamins, and other essential components such as electrolytes and trace elements. In general, PN solutions are commercially prepared as separate groups of components, i.e., as an amino acid solution or with a dextrose, electrolyte, mineral solution, and then mixed together before administration at a ratio to give final nutrient concentrations to meet the optimal nutritional requirements for the patient. Typically, the present practice of PN provides a solution of amino acids which can be mixed with a solution of dextrose (i.e., carbohydrate) and other necessary supplements.

Representative compositions for PN solutions are well known and many commercial preparations are available. PN is a solution that contains fluids, carbohydrates, electrolytes, proteins, amino acids, minerals, vitamins, and trace minerals. PN is administered concurrently with an intravenous lipid emulsion or as a part of total nutrient admixture that provides essential fatty acids. These lipid emulsions can comprise a vegetable oil, such as soybean oil or safflower oil, an emulsifying agent such as egg phospholipids, glycerol, and water. Thus, the fatty acid content is comprised primarily of the essential omega-6 fatty acids with some omega-9 and omega-3 poly-unsaturated fatty acids.

PN amino acid solutions are usually provided as about 5-15% solutions of amino acids and can be delivered to the patient as approximately 1-5% of protein nutrient mixture. The 20 common amino acids can be included in such solutions although some PN products are limited to the essential and semi-essential amino acids as deemed appropriate for the disease state of the patient. The amino acid solutions can also include ornithine, citrulline and taurine. For example, in pediatric formulations, 17 of the 20 common amino acids are generally included, with omission of cysteine, glutamine, and asparagine (because of their instability in solution) and addition of taurine. An example of a PN amino acid solution is described in U.S. Pat. No. 4,491,589 which is incorporated herein by reference.

As used herein, “enteral”, “enteral nutrition”, “enteral feeding”, and “enteral administration” are used interchangeably and refer to the administration of nutrients within, or by way of, the intestine or gastrointestinal tract, especially as distinguished from parenteral administration. Enteral nutrition can comprise oral feeding or administration, i.e. by mouth, or direct administration of nutrients to the gastrointestinal tract by way of feeding tube, e.g. nasogastric (NG), orogastric (OG), transpyloric, percutaneous endoscopic gastrostomy or gastrostomy (G)-tube.

As used herein, “parenteral nutrition associated liver disease”, or “PNALD”, also known as PN induced liver disease, cholestatic liver disease, and intestinal failure associated liver disease, are used interchangeably and refer to the condition or disease of the liver which is associated with or induced by PN. PNALD can include both biochemical, i.e., elevated serum aminotransferase, bilirubin, and alkaline phosphatase, and histologic alterations such as steatosis, steatohepatitis, lipidosis, cholestasis, fibrosis, and cirrhosis. PNALD can be progressive and worsen with the course of PN administration. In some embodiments, a subject is diagnosed with PNALD when the subject has direct bilirubin concentrations of >2 mg/dL and/or elevated transaminases, e.g. AST and ALT.

As used herein, “short bowel syndrome” or “SBS” is used interchangeably and refers to a condition due to loss of some of a subject's small intestine removed because of surgical removal due to disease of the small intestine. In some embodiments, “short bowel syndrome” or “SBS” is used interchangeably and refers to a condition due to loss of half or more of a subject's small intestine removed because of surgical removal due to disease of the small intestine. Common reasons for removing part of the small intestine include surgery for Crohn's disease, ulcerative colitis, necrotizing enterocolitis (NEC), an infectious inflammatory disease of premature newborns, intestinal atresia, failure of development of part of the intestine, volvulus, which occurs when the bowel gets twisted and the blood supply is impaired, and gastroschesis, omphalocele, and Hirschprungs disease. SBS is also known to those of ordinary skill in the art as “short gut” or “effective short gut”.

II. General Considerations

The presently disclosed subject matter provides compositions comprising omega-3 fatty acids and uses thereof. Intravenous fish oil has shown promise in the treatment of parenteral nutrition (PN) associated liver disease (PNALD). However, given the assumption that subjects on PN, particularly infants, are generally intolerant to enteral feeding, enteral administration of omega-3 fatty acids, e.g. fish oil compositions and algal sourced oil compositions, has not been evaluated for treatment of PNALD prior to the instant disclosure. Surprisingly, the presently disclosed subject matter shows that PNALD can be at least partially, and in some cases completely reversed in some infants receiving enteral fish oil therapy, suggesting that enteral omega-3 fatty acid administration is a treatment for PNALD. Based upon this finding, omega-3 fatty acid compositions and treatment regimens are provided for the treatment and prevention of liver diseases.

III. Compositions of the Presently Disclosed Subject Matter

Provided herein in some embodiments are compositions comprising an emulsion or suspension of omega-3 fatty acids using docosahexanoic acid (DHA) and eicosapentaenoic acid (EPA) as either the triglyceride or as the ethyl ester form. In some embodiments, the DHA and EPA are sourced from either fish or algal materials. In some embodiments, a stable emulsion/suspension of omega-3 fatty acids provided herein is suitable for oral or feeding tube administration, i.e. enteral administration. In some embodiments, the omega-3 fatty acid compositions comprise a de-odorized omega-3 fatty acid composition. In some embodiments, flavoring and/or masking agents can be employed to enhance the taste, smell and/or palatability of the product.

In some embodiments, omega-3 fatty acid compositions of the presently disclosed subject matter are in the ethyl ester form, or substantially in the ethyl ester form. In some embodiments, omega-3 fatty acid compositions of the presently disclosed subject matter are in the triglyceride form, or substantially in the triglyceride form. In some embodiments, algal sourced omega-3 fatty acid compositions of the presently disclosed subject matter are substantially in the triglyceride, diglyceride, or ethyl ester form. In some embodiments, algal sourced omega-3 fatty acid compositions of the presently disclosed subject matter are substantially in the triglyceride form. In some embodiments, algal sourced omega-3 fatty acid compositions of the presently disclosed subject matter are substantially in the triglyceride, diglyceride, or ethyl ester form, wherein the composition is 35% DHA (wt/wt) with little to no EPA.

The term “substantially”, as used herein to describe the compositions of the presently disclosed subject matter, refers to the make-up of the composition. For example, in some embodiments, a composition of the presently disclosed subject matter that is substantially in the triglyceride form, substantially in the ethyl ester form, or substantially in the diglyceride form, refers to a composition that is at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99%, triglyceride, ethyl ester, or diglyceride respectively.

In some embodiments, the presently disclosed subject matter provides for a palatable omega-3 fatty acid composition comprising DHA and

EPA for enteral administration. In some embodiments, the omega-3 fatty acid composition comprises fish oil.

In some embodiments, a particular dosage of DHA and EPA can be incorporated into an oil-in-water emulsion or a powder-in-liquid suspension. In some embodiments, the omega-3 fatty acid compositions of the presently disclosed subject matter can be designed to achieve a desired formulation that most closely matches an effective dosage of DHA and EPA in an acceptable volume. In some embodiments, the DHA:EPA ratio can range from approximately 1:3 to 3:1. In some embodiments, the DHA:EPA ratio can range from approximately 1:2.5 to 2.5:1; 1:2 to 2:1, 1:1.5 to 1.5:1, and 1:1. In some embodiments, the omega-3 fatty acid compositions can be formulated to provide an effective dosage of DHA and EPA of approximately 0.1 mg/kg/d to 1 g/kg/d, i.e. 0.1 milligrams to 1 gram of DHA and EPA per kilogram of body weight per day. In some embodiments, the omega-3 fatty acid compositions can be formulated to provide an effective dosage of DHA and EPA of approximately 1 mg/kg/d to 500 mg/kg/d; 10 mg/kg/d to 450 mg/kg/d; 20 mg/kg/d to 400 mg/kg/d; 30 mg/kg/d to 350 mg/kg/d; 40 mg/kg/d to 300 mg/kg/d; 50 mg/kg/d to 250 mg/kg/d; 60 mg/kg/d to 200 mg/kg/d; 70 mg/kg/d to 150 mg/kg/d; or 80 mg/kg/d to 100 mg/kg/d. In some embodiments, the omega-3 fatty acid compositions can be formulated to provide the appropriate dosages of DHA and EPA to meet a therapeutic endpoint of resolving or preventing PNALD in a convenient to use volume.

In some embodiments, one, two, three or more sources of oil and/or powder can be used for the omega-3 fatty acid compositions. In some embodiments, the sources of raw material for the omega-3 fatty acid compositions of the presently disclosed subject matter can comprise fish oil or fish products, algal materials, or any other known sources of omega-3 fatty acids. In some embodiments, fish oil used as a source for the omega-3 fatty acid compositions can be derived from any fish including, but not limited to, menhaden, herring, mackerel, cod, caplin, tilapia, tuna, sardine, pacific saury, salmon, and krill.

Fish oils can contain DHA and EPA in relatively high concentrations. Since isolation of these acids from natural products and the chemical synthesis is costly, fish oils are considered relatively inexpensive sources of these essential fatty acids. Methods of extracting and refining fish oils are known in the art.

In some embodiments, algae can be a source of omega-3 fatty acids. A number of algal sources of omega-3 fatty acids are well known in the art. These sources are also relatively inexpensive sources of these essential fatty acids. Methods of extracting and refining these algal oils are well known in the art.

In some embodiments, flavoring and/or masking agents can be employed in the omega-3 fatty acid compositions to minimize the odor associated with fish oil products, which can be significant. In some embodiments, the compositions can be formulated from a de-odorized marine or algal source raw material. In some embodiments, the omega-3 fatty acid compositions further comprise flavoring and/or masking agents to enhance the taste, smell and/or palatability of the product. In some embodiments, the omega-3 fatty acid compositions can be developed based on the acceptability of smell, flavor, and texture by care-givers and/or patients. Accordingly, as used herein, “palatable”, “palatability”, and variations thereof are used interchangeably and refer to the sensory properties of a compound, including but not limited to taste, flavor, smell, odor, texture, or any combination thereof, as perceived by a care-giver or patient. In some embodiments, the omega-3 fatty acid compositions provided herein comprise enhanced palatability or are more palatable than compositions not comprising flavoring and/or masking agents. In some embodiments, the omega-3 fatty acid compositions provided herein are palatable, i.e. acceptable by either care-givers or patients, whereas other compositions, e.g. fish oil, is considered unpalatable.

Non-limiting examples of omega-3 fatty acid compositions and methods of making the same can be found in Examples 8-10. By way of example and not limitation, an omega-3 fatty acid composition of the presently disclosed subject matter can comprise a 50:50 (vol/vol) oil-in-water emulsion in which the oil is a fish oil mixture of EPA and DHA. In some embodiments, a composition of the presently disclosed subject matter can also comprise about 15% (wt/vol) sugar, about 1.5% (wt/vol) soy lecithin, about 0.5% carrageenan, about 1.5% (vol/vol) flavoring, and/or about 1.0% masking agent.

By way of example and not limitation, an omega-3 fatty acid composition of the presently disclosed subject matter can comprise a 50:50 (vol/vol) oil-in-water emulsion in which the oil is a steam de-odorized fish oil mixture of EPA and DHA. In some embodiments, a composition of the presently disclosed subject matter can also comprise about 12.5% (wt/vol) sugar, about 1.5% (wt/vol) soy lecithin, and/or about 1.0% (vol/vol) flavoring.

By way of example and not limitation, an omega-3 fatty acid composition of the presently disclosed subject matter can comprise a 40:60 (vol/vol) oil-in-water emulsion in which the oil is an algal oil mixture of EPA and DHA. In some embodiments, a composition of the presently disclosed subject matter can also comprise about 12.5% (wt/vol) sugar, about 1.5% (wt/vol) soy lecithin, with or without about 0.5% carrageenan and about 1.0% (vol/vol) flavoring.

In some embodiments omega-3 fatty acid compositions of the presently disclosed subject matter can be made by preparing an emulsion. In some embodiments, an emulsion can be achieved by heating water in a beaker while stirring. In some embodiments the water can be heated to about 40° C. to about 70° C. In some embodiments the water can be heated to about 50° C. to about 60° C. In some embodiments the water can be heated to about 50° C., in some embodiments to about 51° C., in some embodiments to about 52° C., in some embodiments to about 53° C., in some embodiments to about 54° C., in some embodiments to about 55° C., in some embodiments to about 56° C., in some embodiments to about 57° C., in some embodiments to about 58° C., in some embodiments to about 59° C., and in some embodiments to about 60° C. In some embodiments, sugar can be dissolved in the water. Similarly, in some embodiments oil can be heated in while stirring. In some embodiments the oil can be heated to about 40° C. to about 70° C. In some embodiments the oil can be heated to about 50° C. to about 60° C. In some embodiments the oil can be heated to about 50° C., in some embodiments to about 51° C., in some embodiments to about 52° C., in some embodiments to about 53° C., in some embodiments to about 54° C., in some embodiments to about 55° C., in some embodiments to about 56° C., in some embodiments to about 57° C., in some embodiments to about 58° C., in some embodiments to about 59° C., and in some embodiments to about 60° C. In some embodiments, soy lecithin and/or carrageenan can be dissolved slowly in the oil. Then, in some embodiments a homogenizer can be positioned in the water to achieve efficient mixing, and the oil mixture can be slowly added to the water while maintaining the temperature of the mixture, e.g. at 50 to 60° C. In some embodiments the speed of mixing can be increased as necessary (as the emulsion forms and viscosity increases) to achieve a complete mixture. After completing the oil addition, in some embodiments the homogenizer can be adjusted to a medium speed and mixing can be continued for at least 30 minutes or as necessary to achieve a stable emulsion. Flavoring and masking agents (if used) can be added to the completed emulsion and mixed for an additional period of time, e.g. five to ten minutes. Alternatively, in some embodiments flavoring and masking agents can be dissolved in the water or oil (depending on solubility) prior to mixing.

In some embodiments, the omega-3 fatty acid compositions of the presently disclosed subject matter can be prepared by combining one or more emulsifying agents with one or more sources of omega-3 fatty acids. Emulsifying agents for this purpose can generally be phospholipids of natural, synthetic or semi-synthetic origin. A variety of suitable emulsifying agents are known in the art. Examples of suitable emulsifying agents include, but are not limited to, egg phosphatidylcholine, egg lecithin, soy lecithin, L-α-dipalmitoyl phosphatidylcholine (DPPC), DL-α-dipalmitoyl phosphatidylethanolamine (DPPE), and dioleoyl phosphatidylcholine (DOPC). In some embodiments, the compositions of the presently disclosed subject matter can comprise between about 0.1% and about 5% (w/v) emulsifying agent, or about 0.5% to 4% (w/v); or 1% to 3% (w/v) emulsifying agent. In some embodiments, the omega-3 fatty acid compositions of the presently disclosed subject matter can comprise additional components such as antioxidants, chelating agents, osmolality modifiers, buffers, neutralization agents, thickening agents, e.g. carrageenins, and the like that improve the stability, uniformity and/or other properties of the emulsion. In some embodiments, one or more antioxidants can be added to the omega-3 fatty acid compositions to prevent the formation of undesirable oxidized fatty acids. By way of example and not limitation, suitable antioxidants can comprise alpha-tocopherol (vitamin E) and tocotrienols.

In some embodiments, the omega-3 fatty acid compositions can comprise a therapeutic agent in addition to the omega-3 fatty acids. A “therapeutic agent” as used herein refers to a physiologically or pharmacologically active substance that produces a localized or systemic effect or effects in the subject to which it is administered and refers generally to drugs, nutritional supplements, vitamins, minerals, enzymes, hormones, proteins, polypeptides, antigens and other therapeutically or diagnostically useful compounds.

In some embodiments, the omega-3 fatty acid compositions of the presently disclosed subject, matter can be in the form of an oil-in-water emulsion, including nano-particle emulsions, or a powder-in-liquid suspension. In some embodiments, a micro-encapsulated powdered preparation can be used as a starting material and formulated into either a suspension or emulsion product. In some embodiments, the omega-3 fatty acid compositions (emulsion and/or suspension) are designed to be administered enterally, e.g. orally. In some embodiments, the omega-3 fatty acid compositions allow for accurate and consistent dosage of omega-3 fatty acids.

IV. Methods of the Presently Disclosed Subject Matter

In some embodiments, the presently disclosed subject matter provides methods for treating or preventing liver disease in subjects receiving PN. In some embodiments the methods comprises enteral administration of an effective amount of an omega-3 fatty acid composition to a subject. In some embodiments, the subjects to be treated are infants and children receiving PN and at risk for PNALD.

In some embodiments, the disease to be prevented or treated is PN associated or induced liver disease. This disease can include both biochemical, i.e., elevated serum aminotransferases, total and direct bilirubin, gamma-glutamyl transpeptidase (GGT), and alkaline phosphatase (alk phos), and histologic alterations such as steatosis, steatohepatitis, lipidosis, cholestasis, fibrosis, and cirrhosis. PNALD can be progressive and worsen with the course of PN administration. All subjects administered PN are susceptible to PNALD. Pediatric subjects administered PN can be particularly susceptible to PNALD. Additional risk factors for this condition include prematurity, low birth weight, very low birth weight, extremely low birth weight, long-term PN use, the lack of concomitant oral intake, sepsis (early on-set and duration of septic events), and multiple operative procedures.

In some embodiments, a method is provided for treating PNALD in a subject, the method comprising: providing a subject with PNALD; and administering to the subject an effective amount of an omega-3 fatty acid composition, wherein the omega-3 fatty acid composition is administered enterally. In some embodiments, the subject is an infant. In some embodiments, the subject is a pre-term infant. In some embodiments, the subject is an infant having a low birth weight, very low birth weight, extremely low birth weight, a low gestational age, short bowel syndrome (SBS), necrotizing enterocolitis (NEC), gastroeschesis, omphelacele, atresias, Hirschprungs disease, and functional short bowel syndrome or any combination thereof. In some embodiments, the subject is receiving PN. In some embodiments, the omega-3 fatty acid composition administered comprises DHA and EPA. In some embodiments, the omega-3 fatty acid composition comprises fish oil, deodorized fish oil, or algal oil. In some embodiments, the effective amount of an omega-3 fatty acid composition comprises a dose of 0.1 g/kg/day to 1 g/kg/day, and in some embodiments a ratio of approximately 1:3 to 3:1 of DHA to EPA. In some embodiments, treating PNALD in a subject comprises reversal of PNALD, wherein PNALD reversal is defined as three consecutive direct bilirubin measurements of less than 2 mg/dL. In some embodiments, PNALD reversal is defined as three consecutive direct bilirubin measurements of less than 2 mg/dL along with clinical correlation, wherein clinical correlation can be defined as decreased jaundice, decreased liver size, decreased liver enzymes, increased enteral tolerance, increased weight gain, improvement in clotting factors, and/or improvement in visceral proteins (e.g., albumin, prealbumin, and/or total protein, and the like).

In some embodiments, a method is provided for preventing PNALD in a subject, the method comprising: providing a subject at risk for developing PNALD; and administering to the subject an effective amount of an omega-3 fatty acid composition, wherein the omega-3 fatty acid composition is administered enterally. In some embodiments, the subject at risk for developing PNALD is any subject receiving PN. In some embodiments, the subject at risk for developing PNALD is an infant receiving PN. In some embodiments, the subject at risk for developing PNALD is a pre-term infant receiving PN. In some embodiments, the subject is an infant having a low birth weight, very low birth weight, extremely low birth weight, a low gestational age, SBS, NEC, gastroeschesis, omphelacele, atresias, Hirschprungs disease, and function short bowel syndrome or any combination thereof. In some embodiments, the omega-3 fatty acid composition administered comprises DHA and EPA. In some embodiments, the omega-3 fatty acid composition comprises fish oil, deodorized fish oil, or algal oil. In some embodiments, the omega-3 fatty acid composition comprises a 50:50 (vol/vol) oil-in-water emulsion in which the oil is a fish oil mixture of EPA and DHA. In some embodiments, the omega-3 fatty acid composition comprises a 50:50 (vol/vol) oil-in-water emulsion in which the oil is a steam de-odorized fish oil mixture of EPA and DHA. In some embodiments, the omega-3 fatty acid composition comprises a 40:60 (vol/vol) oil-in-water emulsion in which the oil is an algal oil mixture of EPA and DHA. In some embodiments, the effective amount of an omega-3 fatty acid composition comprises a dose of 0.1 g/kg/day to 1 g/kg/day, and in some embodiments, a ratio of approximately 1:3 to 3:1 of DHA to EPA. In some embodiments, the effective amount of an omega-3 fatty acid composition comprises a DHA:EPA ratio ranging from approximately 1:2.5 to 2.5:1; 1:2 to 2:1, 1:1.5 to 1.5:1, and 1:1.

In some embodiments, the presently disclosed subject matter provides methods for improving a subject's ability to receive nutritional support enterally. In some embodiments, enteral administration of an effective amount of an omega-3 fatty acid composition to a subject receiving PN can substantially shorten the time the subject requires PN and advance the subject's tolerance of enteral feeding. In some embodiments, shortening the PN regimen and advancing enteral tolerance can prevent, minimize and/or treat PNALD. In some embodiments, the methods comprise enteral administration of an effective amount of an omega-3 fatty acid composition to a subject. In some embodiments, the subjects to be treated are infants receiving PN and at risk for PNALD.

In some embodiments, a method is provided for advancing enteral tolerance in a subject receiving parenteral nutrition, the method comprising: providing a subject receiving parenteral nutrition; and administering to the subject an effective amount of an omega-3 fatty acid composition, wherein the omega-3 fatty acid composition is administered enterally. In some embodiments, the subject is an infant receiving parenteral nutrition. In some embodiments, the subject is a pre-term infant receiving parenteral nutrition. In some embodiments, the subject is an infant having a low birth weight, very low birth weight, extremely low birth weight, a low gestational age, SBS, NEC, gastroeschesis, omphelacele, atresias, Hirschprungs disease, and function short bowel syndrome or any combination thereof. In some embodiments, the omega-3 fatty acid composition administered comprises DHA and EPA. In some embodiments, the omega-3 fatty acid composition comprises fish oil, deodorized fish oil, or algal oil. In some embodiments, the omega-3 fatty acid composition comprises a 50:50 (vol/vol) oil-in-water emulsion in which the oil is a fish oil mixture of EPA and DHA. In some embodiments, the omega-3 fatty acid composition comprises a 50:50 (vol/vol) oil-in-water emulsion in which the oil is a steam de-odorized fish oil mixture of EPA and DHA. In some embodiments, the omega-3 fatty acid composition comprises a 40:60 (vol/vol) oil-in-water emulsion in which the oil is an algal oil mixture of EPA and DHA. In some embodiments, the effective amount of an omega-3 fatty acid composition comprises a dose of 0.1 g/kg/day to 1 g/kg/day, and in some embodiments a ratio of approximately 1:3 to 3:1 of DHA to EPA. In some embodiments, advancing enteral tolerance comprises an enhanced ability for the subject to tolerate enteral feeding as compared to a subject not administered an effective amount of an omega-3 fatty acid composition. In some embodiments, advancing enteral tolerance results in the subject requiring PN for a reduced period of time as compared to a subject not administered an effective amount of an omega-3 fatty acid composition. In some embodiments, the reduced period of time that the subject requires PN can comprise a period of time of about 1, 2, 3, 4, 5, 6, 7, 8, 9, 10, 15, 20, 25, 30, 35, 40, 45, 50, 75, or 100 fewer days or more. In some embodiments, the reduced period of time that the subject requires PN can comprise a period of time of about 1, 2, 3, 4, 5, 6, 7, 8, 9, 10, 15, 20, 25, 30, 35, 40, 45, 50, 75, or 100 weeks or more.

In some embodiments omega-3 fatty acids and compositions comprising omega-3 fatty acids can be anti-inflammatory or have anti-inflammatory properties. Therefore, in some embodiments, a method is provided for treating or preventing diseases with an inflammatory component, the method comprising: providing a subject having or at risk for developing a disease with an inflammatory component; and administering to the subject an effective amount of an omega-3 fatty acid composition. In some embodiments the omega-3 polyunsaturated fatty acid compositions provided herein can be used for the prevention and/or treatment of any disease or condition with an inflammatory component, including but not limited to, pediatric or adult inflammatory bowel disease, cystic fibrosis, critical illness, burns, metabolic syndrome, obesity, malignancy related weight loss, bipolar disorder, and cardiovascular disease. In some embodiments, the omega-3 fatty acid composition administered comprises DHA and EPA. In some embodiments, the omega-3 fatty acid composition comprises fish oil, deodorized fish oil, or algal oil. In some embodiments, the omega-3 fatty acid composition comprises a 50:50 (vol/vol) oil-in-water emulsion in which the oil is a fish oil mixture of EPA and DHA. In some embodiments, the omega-3 fatty acid composition comprises a 50:50 (vol/vol) oil-in-water emulsion in which the oil is a steam de-odorized fish oil mixture of EPA and DHA. In some embodiments, the omega-3 fatty acid composition comprises a 40:60 (vol/vol) oil-in-water emulsion in which the oil is an algal oil mixture of EPA and DHA.

Inflammatory diseases and diseases with an inflammatory component, including but not limited to, pediatric or adult inflammatory bowel disease, cystic fibrosis, critical illness, burns, metabolic syndrome, obesity, malignancy related weight loss, bipolar disorder, and cardiovascular disease, are known in the art. See, for example, Shimizu et al., 2003; Panchaud et al., 2006; Pontes-rruda et al., 2006; Yang et al., 2010; Skulas-Ray et al., 2010; Noel et al., 2010; Murff et al., 2010; Clayton et al., 2008; Burrows et al., 2011. Each of the forgoing references are incorporated herein in their entireties.

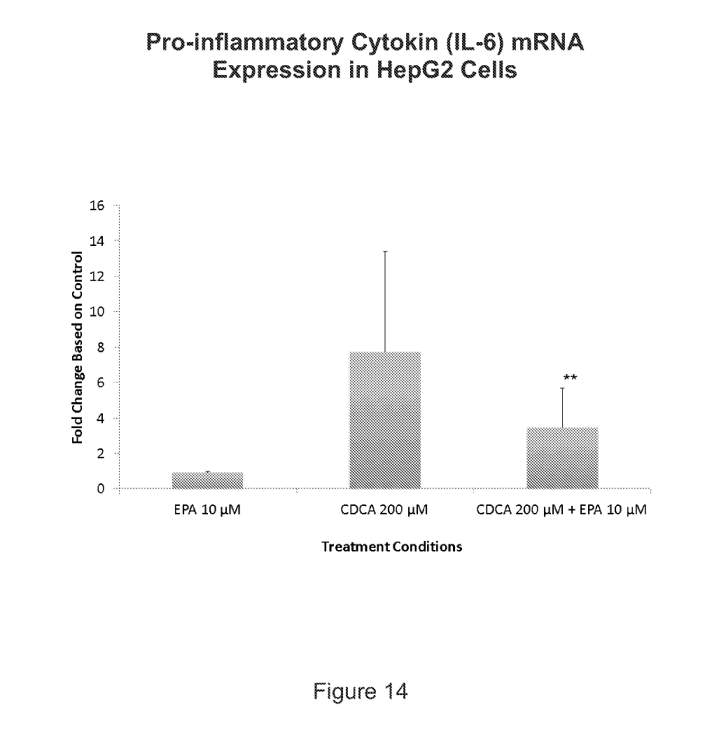

Although not intended to be bound or limited by any particular theory or mechanism of action, in some embodiments of the presently disclosed methods, including treating or preventing diseases with an inflammatory component, anti-inflammatory effects can be achieved through the regulation of pro-inflammatory cytokine (IL-6) mRNA expression. In some embodiments, treatment with EPA and/or DHA can attenuate pro-inflammatory cytokine (IL-6) mRNA expression. In some embodiments, omega-3 fatty acids such as EPA and/or DHA can treat or prevent diseases with an inflammatory component. See, Example 13 and FIG. 14:

Although not intended to be bound or limited by any particular theory or mechanism of action, in some embodiments of the presently disclosed methods, including methods of treating PNALD in a subject, preventing PNALD in a subject, improving a subject's ability to receive nutritional support enterally, advancing enteral tolerance in a subject receiving parenteral nutrition, and/or treating or preventing diseases with an inflammatory component, the notable improvement in PNALD and EN advancement in subjects administered omega-3 fatty acid compositions can be achieved through the attenuation of apoptosis induced by high levels of retained hydrophobic bile acids. In some embodiments, treatment with EPA alone and DHA alone can result in attenuation of apoptosis. In some embodiments, the combination of EPA and DHA can result in a synergistic attenuation of bile acid-induced hepatocellular apoptosis, which can have a greater attenuating effect than treatment with EPA and DHA separately. As such, in some embodiments, the presently disclosed subject matter provides methods of attenuating bile acid-induced hepatocellular apoptosis, comprising administering to a subject in need thereof compositions comprising EPA, DHA, and/or a combination of EPA and DHA.

To elaborate, the etiology of PNALD is not well understood and likely multi-factorial and possibly attributed to immature bile secretion, inflammation, oxidative stress, infection, nutrient deficiencies, and/or toxic components in parenteral products including lipids or amino acids. Lipophilic bile acids, which are often increased in PNALD, are known to cause cellular apoptosis. Many lipophilic bile acids have been shown to induce apoptosis in both cellular and animal models (Higuchi et al., 2003; Amaral et al., 2007; Yang et al., 2007; Reinehr et al., 2004; Gumpricht et al., 2005; Bern et al., 2006; Park et al., 2008). Apoptosis occurs by activation of death receptors (DR) located on the cell surface. There are at least six known death receptors, but the two that have been shown to be involved in apoptosis in the liver are Fas and tumor necrosis factor-associated apoptosis-inducing ligand receptor 2 (TRAIL-R2; Higuchi et al., 2001). Apoptosis occurs via different pathways depending on cell type. Bile acid-induced apoptosis is thought to occur via the Fas and TRAIL-R2 death receptors (Higuchi et al., 2001).

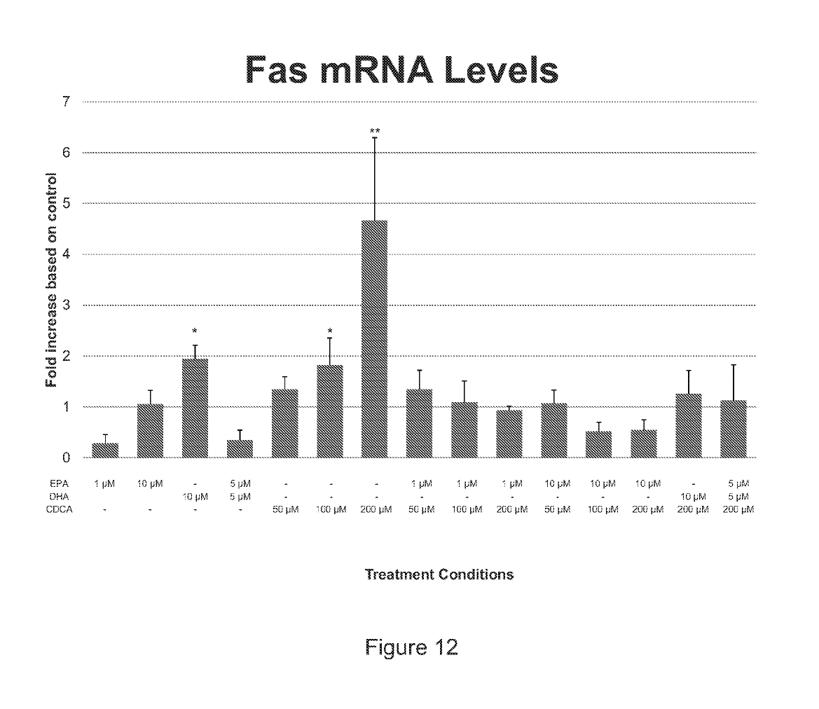

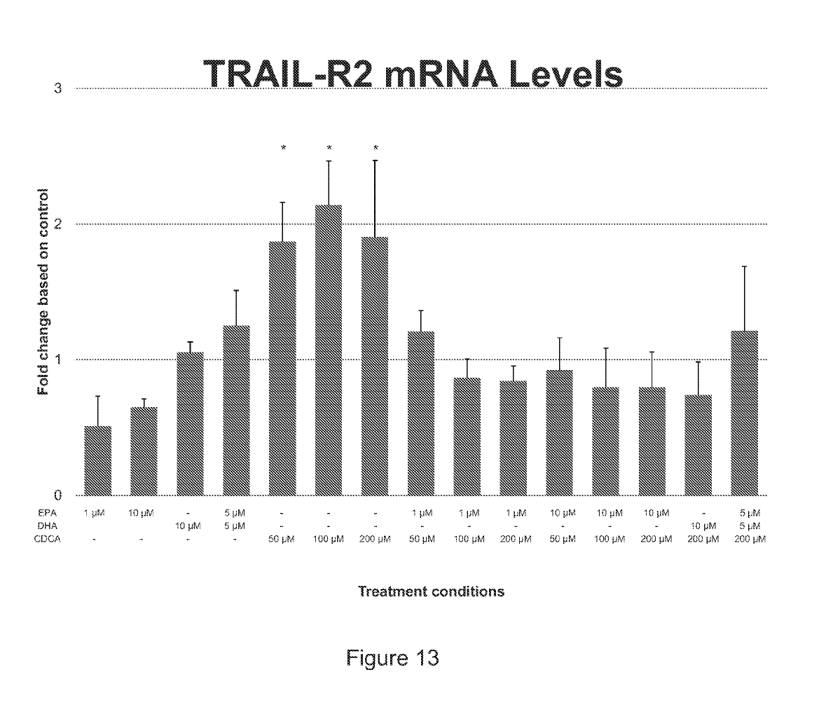

Although not intended to be bound or limited by any particular theory or mechanism of action, in some embodiments of the presently disclosed methods, the omega-3 fatty acid attenuation of apoptosis induced by high levels of retained hydrophobic bile acids can be achieved by regulating expression of Fas and TRAIL-R2 mRNA. In some embodiments, treatment with EPA and/or DHA can result in attenuation of apoptosis by attenuating the up-regulation of expression of Fas and TRAIL-R2 mRNA by high levels of retained hydrophobic bile acids. As such, in some embodiments, the presently disclosed subject matter provides methods of attenuating bile acid-induced hepatocellular apoptosis, comprising administering to a subject in need thereof compositions comprising EPA, DHA, and/or a combination of EPA and DHA. See, Example 12 and FIGS. 12 and 13.

In some embodiments, the omega-3 fatty acid composition is administered to a subject for a period of time sufficient to treat PNALD. In some embodiments, the administration is for a period of time sufficient to decrease bilirubin in a subject below 2 mg/dL. In some embodiments, the administration is for a period of time sufficient to decrease transaminases, e.g. aspartate aminotransferase (AST) and alanine aminotransferase (ALT), GGT, and alk phos in a subject. In some embodiments, the administration is for a period of time sufficient to regulate pro-inflammatory cytokine (IL-6) mRNA expression. In some embodiments, the administration is for a period of time sufficient to regulate expression of Fas and TRAIL-R2 mRNA. In some embodiments, the omega-3 fatty acid compositions of the presently disclosed subject matter are administered to a subject in conjunction with or in parallel with PN. In some embodiments, the omega-3 fatty acid compositions of the presently disclosed subject matter are administered for a period of time before, during and/or after the initiation of PN. In some embodiments, the administration is for a period of time sufficient to reverse PNALD, wherein PNALD reversal is defined as three consecutive direct bilirubin measurements <2 mg/dL and clinical correlation. In some embodiments, the administration comprises a period of time of about 1, 2, 3, 4, 5, 6, 7, 8, 9, 10, 15, 20, 25, 30, 35, 40, 45, 50, 75, or 100 days or more. In some embodiments, the administration comprises a period of time of about 1, 2, 3, 4, 5, 6, 7, 8, 9, 10, 15, 20, 25, 30, 35, 40, 45, 50, 75, or 100 weeks or more. In some embodiments, the administration comprises a period of time of about 1, 2, 3, 4, 5, 6, 7, 8, 9, 10, 15, 20, 25, 30, 35, 40, 45, 50, 75, or 100 months or more. In some embodiments, the administration comprises a period of time of about 1, 2, 3, 4, 5, 6, 7, 8, 9, 10, 15, 20, 25, 30, 35, 40, 45 or 50 years or more. In some embodiments, the administration comprises a period of time extending over days, weeks, months or years of chronic therapy to prevent future occurrences.

As used herein, the terms “effective amount” and “therapeutically effective amount” are used interchangeably and mean a dosage sufficient to provide treatment for the disease state being treated. In some embodiments, an effective amount of omega-3 fatty acid composition can comprise a dose of 0.1 g/kg/day to 1 g/kg/day, and in some embodiments a ratio of approximately 1:3 to 3:1 of DHA to EPA. In some embodiments, an effective amount of omega-3 fatty acid composition can comprise an amount sufficient to decrease direct bilirubin in a subject below 2 mg/dL. In some embodiments, an effective amount of omega-3 fatty acid composition can comprise an amount sufficient to decrease transaminases, e.g. aspartate aminotransferase (AST), alanine aminotransferase (ALT), GGT, and alk phos in a subject. In some embodiments, an effective amount of omega-3 fatty acid composition can comprise an amount sufficient to reverse PNALD, wherein PNALD reversal is defined as three consecutive direct bilirubin measurements <2 mg/dL.

In some embodiments, the subject to be treated comprises any subject at risk for developing PNALD and/or receiving PN. In some embodiments, the subject to be treated comprises an infant. In some embodiments, the subject is an infant having a low birth weight, very low birth weight, extremely low birth weight, a low gestational age, SBS, NEC, gastroeschesis, omphelacele, atresias, Hirschprungs disease, and function short bowel syndrome or any combination thereof. In some embodiments, an infant of low gestational age can be an infant of less than 38 weeks of gestational age. In some embodiments, an infant of low gestational age can be an infant born pre-term. In some embodiments, an infant of low birth weight (LBW) can be an infant weighing <2,500 grams at birth. In some embodiments, an infant of very low birth weight (VLBW) can be an infant weighing <1,500 grams at birth. In some embodiments, an infant of extremely low birth weight (ELBW) can be an infant weighing <1,000 grams at birth.

In some embodiments, the subject is intolerant of enteral feeding. In some embodiments, the subject has PNALD. In some embodiments, the subject to be treated has PNALD, comprising direct bilirubin concentrations of >2 mg/dL and/or elevated transaminases, e.g. AST, ALT, GGT, and alk phos. In some embodiments, the subject is any human subject receiving PN and at risk for developing PNALD.

The subjects treated in the presently disclosed subject matter in its many embodiments are desirably a human subject, although it is to be understood the methods described herein are effective with respect to all vertebrate species, which are intended to be included in the term “subject.” In some embodiments the subject is warm-blooded vertebrate.

More particularly, provided herein is the treatment of mammals, such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided herein is the treatment of birds, including the treatment of those kinds of birds that are endangered, kept in zoos or as pets, as well as fowl, and more particularly domesticated fowl, i.e., poultry, such as turkeys, chickens, ducks, geese, guinea fowl, and the like, as they also are of economic importance to humans. Thus, embodiments of the methods described herein include the treatment of livestock, including, but not limited to, domesticated swine (pigs and hogs), ruminants, horses, poultry, and the like.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Research Design

### Experimental Design for Examples 1-6

Infants with short bowel syndrome (SBS) received approximately 90-100 kcal/kg/day via parenteral nutrition (PN) comprising approximately 10-12% protein, 10-30% lipid, and 60-70% carbohydrate. Enteral nutrition (EN) was initiated slowly, starting with low volume trophic feedings which were gradually titrated as tolerated to goal feedings. Caloric intakes were adjusted based on weight gain, linear growth, and enteral feeding tolerance. Standard of care treatment for parenteral nutrition associated liver disease (PNALD) included providing appropriate macro and micronutrients, the initiation and advancement of EN as tolerated, cyclic PN to provide a PN free period each day, and the use of ursodiol 30 mg/kg/day (De Marcho et al., 2006).

Six infants with PNALD who were receiving a combination of PN and EN were supplemented with enteral fish oil and there was no change in soybean based parenteral lipid doses. PNALD diagnosis was based on a direct bilirubin of >2 mg/dL, increased transaminases, and physical exam. Improvement or progression of liver disease was evaluated based on clinical presentation, transaminases, and direct bilirubin concentration. Patient demographic data is shown in Table 1.

TABLE 1
Patient Demographic Data

| Patient | 1 | 2 | 3 | 4 | 5 | 6 |
|---|---|---|---|---|---|---|
| Birth weight (grams) | 1053 | 692 | 1425 | 1001 | 626 | 600 |
| Gestational age | 33 | 31 | 30 | 28 | 27 | 26 |
| (weeks) | | | | | | |
| Primary diagnosis | NEC | Small | NEC | NEC | NEC | NEC |
| | | bowel | | | | |
| | | obstruction | | | | |
| Post-operative | 45-50 | 35 | 20 | 60 | 32 | 8 |
| total small | | | | | | |
| bowel length (cm) | | | | | | |
| Age at onset of | 4 | 3 | 4 | 2 | 8 | 4 |
| PNALD (weeks) | | | | | | |

Enteral fish oil dosing and product varied among patients. The maximum daily dose of fish oil was set at 1 g/kg/day. The individual patient dose was left up to the discretion of the clinician on service at the time and doses were rounded to the nearest capsule, thus there was variability in doses among the patients reviewed. In order to administer the contents of the capsule a nurse withdrew the approximately 1 ml of fish oil from the gelatin capsule shell and the liquid was administered via mouth, nasogastric (NG), or gastrostomy (G)-tube. Due to the variability in over-the-counter dietary supplements, the last two patients were switched to LOVAZA® (Reliant Pharmaceuticals Inc., Liberty Corner, N.J., United States of America), a commercially available prescription only liquid filled capsule that contains purified fish oil with greater amounts of eicosapentaenoic acid (EPA) and docosahexaenoic acid (DHA) compared to over-the-counter (OTC) supplements (See, e.g., Table 2). Compliance with the enteral fish oil regimen was assessed based on interviews with the patient's parents for outpatients and evaluation of medication administration records for inpatients. See Table 2 for a comparison of commercially available fish oil products. Fish oil dosing and response to therapy, including EN and soybean oil based intravenous fat emulsion (IVFE) provision, are shown in Table 3.

TABLE 2
Fish Oil Product Comparison (amounts given as mg per 1 g of
each product)

| | | NATURE | NATURE'S | |
|---|---|---|---|---|
| | LOVAZA ® | MADE ® | BOUNTY ® | OMEGAVEN ™ |
| | (Rx only) | (OTC) | (OTC) | (IV) |
| Linoleic | — | — | 10 | 10-70 |
| α-Linolenic | — | — | 300 | <20 |
| EPA | 465 | 160 | 180 | 125-282 |
| DHA | 375 | 320 | 120 | 144-309 |
| Arachidonic | — | — | — | 10-40 |
| acid | | | | |
| Glycerol | — | — | — | 250 |
| Egg | — | — | — | 120 |
| phospholipid | | | | |
| tocopherol | 4 | — | 1 | 1.5-2.9 |

— Signifies that the information is not available from the manufacture
DHA: docosahexaenoic acid
EPA: eicosapentaenoic acid

TABLE 3
Nutrition, Treatment, and Outcome Data

| Patient | 1 | 2 | 3 | 4 | 5 | [symbol] |
|---|---|---|---|---|---|---|
| Peak total bilirubin (mg/dL)* | 13.6 | 16.5 | 15 | 21.7 | 16.8 | 9 [symbol] |
| Age at initiation of fish oil (months) | 11 | 9 | 6 | 6 | 7 | [symbol] |
| Patient status at initiation of fish oil | Outpatient | Inpatient | Inpatient | Inpatient | Inpatient | Inpa [symbol] |
| Dose of fish oil (g/kg/day) | 0.15 | 0.87 | 1 | 0.8 | 0.6 | 0 [symbol] |
| Dose of fish oil (g/day) | 1 | 3 | 6 | 3 | 3 | [symbol] |
| Fish oil product | NATURE | NATURE'S | NATURE'S | NATURE'S | LOVAZA ® | LOV [symbol] |
| | MADE ® | BOUNTY ® | BOUNTY ® | BOUNTY ® | | |
| Dose of IVFE (g/kg/day)^{†} | 0.9 | 0.6 | 2 | 1 | 0.8 | 1 [symbol] |
| Total nutrition PN + EN | 100 | 120 | 115 | 105 | 115 | 1 [symbol] |
| (kcal/kg/day) | | | | | | |
| % EN at initiation of fish oil | 37% | 56% | 94%^{‡} | 100%^{‡} | 25% | 2 [symbol] |
| EN product | ELECARE ® | ELECARE ® | ELECARE ® | NEOCATE | ELECARE ® | ELE [symbol] |
| Ratio of intake of total omega-3 to | 1:20 | 1:5 | 1:5 | 1:8 | 1:5 | 1 [symbol] |
| omega-6 fatty acids at initiation of | | | | | | |
| fish oil | | | | | | |
| Total bilirubin at initiation of fish oil | 7.9 | 7.2 | 6.2 | 9.5 | 8.6 | 9 [symbol] |
| (mg/dL) | | | | | | |
| AST/ALT at initiation of fish oil | 218/119 | 132/95 | 206/169 | 398/232 | 233/113 | 173 [symbol] |
| (units/L) | | | | | | |
| Serum triglyceride at initiation of | 132 | 240 | 73 | 118 | 146 | 1 [symbol] |
| fish oil (mg/dL) | | | | | | |
| Reversal of PNALD^{§} | Yes | Yes | Yes | No | Yes | N [symbol] |
| Time from initiation of fish oil until | 2 | 4 | 6 | N/A | 6 | N [symbol] |
| reversal (weeks) | | | | | | |
| Total bilirubin 6 weeks after | 0.5 | 1.5 | 0.7 | 5.3 | 2 | [symbol] |
| initiation of fish oil (mg/dL) | | | | | | |
| AST/ALT 6 weeks after initiation of | 35/65 | 102/65 | 89/65 | 199/91 | 109/117 | 144 [symbol] |
| fish oil (units/L) | | | | | | |
| Serum triglyceride 6 weeks after | 41 | 67 | 61 | 76 | 77 | 7 [symbol] |
| initiation of fish oil (mg/dL) | | | | | | |
| % EN at reversal of PNALD | 41% | 100% | 35% | N/A | 68% | N [symbol] |

*Peak total bilirubin occurred after the time period of physiologic jaundice
^{†}A soybean oil based IVFE was used in all patients
^{‡}While these patients were at or near goal EN initially, both patients had a long history of EN intolerance and poor weight gain which resulted in varying amounts of EN given during the time of enteral fish oil supplementation.
^{§}Reversal is defined as 3 consecutive bilirubin measurements <2 mg/dL
NEC: necrotizing enterocolitis
PNALD: parenteral nutrition associated liver disease
IVFE: intravenous fat emulsions
EN: enteral nutrition
AST: aspartate aminotransferase
ALT: alanine aminotransferase [symbol]
indicates data missing or illegible when filed

### Example 1

### Case Report: Patient 1

A male born at 33 weeks gestation developed necrotizing enterocolitis (NEC) that was medically managed with nil per os (NPO, or nothing by mouth), PN and antibiotics. At four weeks of age, patient 1 was transferred to the Le Bonheur Children's Medical Center (Memphis, Tenn., United States of America) where he underwent resection of three areas of small intestine leaving approximately 45-50 cm of small bowel and an intact ileocecal valve. At the time of arrival to Le Bonheur Children's Medical Center, patient 1 had increased direct bilirubin concentrations suggestive of PNALD. Postoperatively patient 1 continued on PN and EN was slowly advanced. PNALD persisted despite the advancement of EN and at nine months of age, patient 1 was discharged home receiving 65% PN cycled over 18 hours with 35% of calories provided enterally. Patient 1 received follow-up care approximately every two weeks in the gastroenterology clinic.

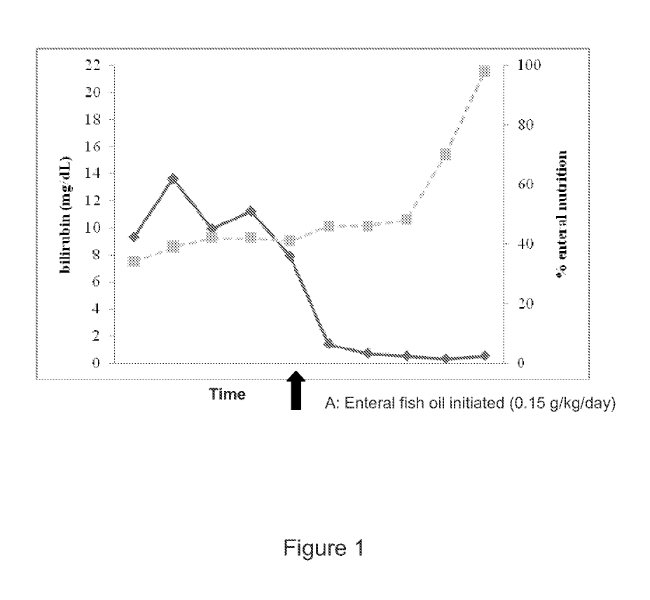

By 11 months of age, patient 1 was receiving approximately 100 kcal/kg/day, 40% of calories from EN and 60% from PN. The total bilirubin was 7.9 mg/dL, and he was prescribed 1 gram of fish oil four times per day (approximately 1 g/kg/day). Two weeks after starting enteral fish oil, the bilirubin had decreased to 1.4 mg/dL. At the clinic visit four weeks after starting fish oil, his previously notable jaundice had resolved and the total bilirubin had decreased to 0.7 mg/dL. Over the next few months, enteral feeding tolerance dramatically improved and by 17 months the patient was off PN and receiving full enteral feeds. At one of his clinic visits during this time it was discovered that his parents were giving him 1 gram of fish oil per day (0.15 g/kg) and not the 4 grams per day originally prescribed. Changes in total bilirubin in connection with EN and enteral fish oil provision are shown in FIG. 1.

### Example 2

### Case Report: Patient 2

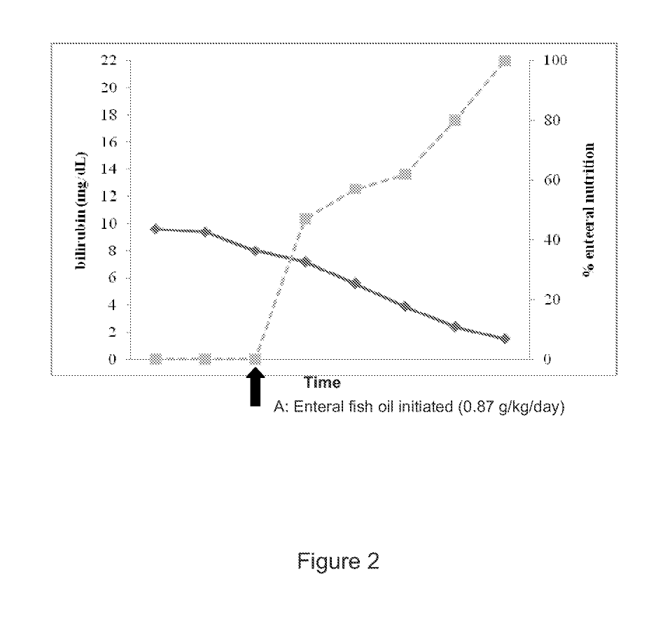

A female born at 31 weeks gestation was admitted on day of life three for the surgical management of small bowel obstruction. She required a small bowel resection and jejunostomy leaving approximately 35 cm of small, bowel with an intact ileocecal valve. Postoperatively patient 2 continued on PN and developed PNALD after three weeks of PN support. After reanastamosis and by nine months of age, EN was slowly being advanced, but her bilirubin remained elevated. Enteral fish oil was initiated at 0.9 g/kg/day, feeding tolerance improved, and PNALD reversed after four weeks (see FIG. 2).

### Example 3

### Case Report: Patient 3

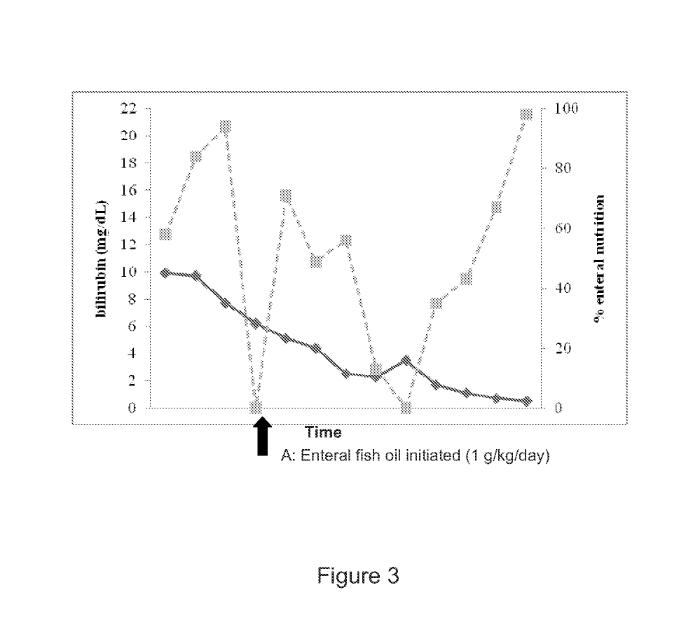

A male born at 30 weeks gestation developed NEC on day of life seven which was medically managed for three weeks. He was transferred to Le Bonheur Children's Medical Center at 4 weeks of age for surgical evaluation and had PNALD upon arrival. He underwent a small bowel resection, jejunostomy and mucous fistula leaving approximately 20 cm of small bowel with an intact ileocecal valve. Postoperatively patient 3 was maintained on PN with supplemental EN. At six months of age and after reanastamosis, EN was slowly being advanced and bilirubin remained increased. Enteral fish oil (1 g/kg/day) was initiated and total bilirubin decreased, but patient 3 continued to experience feeding intolerance. Six weeks after initiation of enteral fish oil, PNALD was completely reversed and feeding tolerance improved. Of note, PNALD resolved despite interruptions in EN due to feeding intolerance (see FIG. 3).

### Example 4

### Case Report: Patient 4

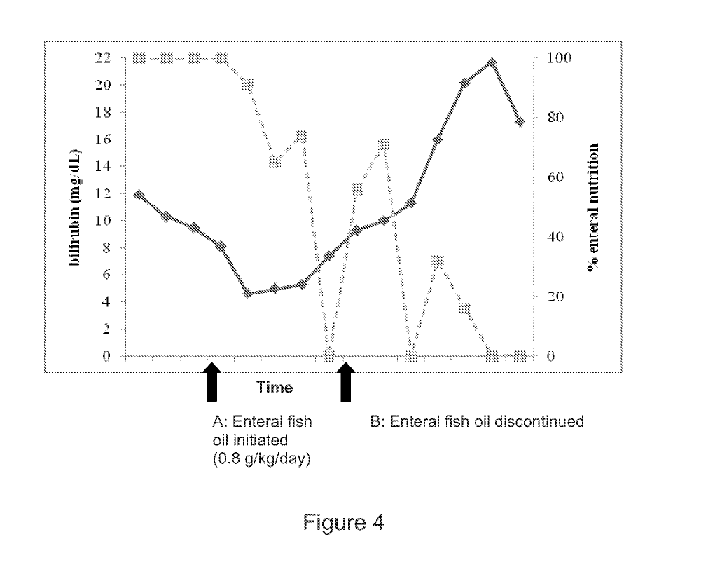

A male born at 28 weeks gestation developed NEC on day of life eight. The patient was transferred to Le Bonheur Children's Medical Center where he underwent ileal resection and jejunostomy placement leaving approximately 60 cm of small bowel and an intact ileocecal valve. Postoperatively patient 4 was maintained on PN and by two weeks of age he had increased direct bilirubin concentration which after negative work-up for other causes was attributed to PNALD. Patient 4 underwent reanastamosis and gastrostomy tube placement at three months of age. The patient continued on PN while EN was slowly being advanced. At six months of age, the patient had progressive PNALD with a bilirubin of 9.5 mg/dL and international normalized ratio (INR) of 1.26. Enteral fish oil was initiated at 1 g/kg/day, and within four weeks, the bilirubin decreased to 4.6 mg/dL. A gastrointestinal tract bleed developed, and he was made NPO requiring the discontinuation of both EN and enteral fish oil and required treatment with four doses of vitamin K. Bilirubin, transaminases, and INR subsequently increased and platelet count decreased over the next several weeks. The patient had blood cultures positive for E. coli and gram positive cocci, and clinical status declined requiring transfer to the intensive care unit where his bleeding was managed with vitamin K, platelets, and fresh frozen plasma. Overwhelming sepsis and hepatic failure ultimately led to the patient's death, although an autopsy was not performed (see FIG. 4).

### Example 5

### Case Report: Patient 5

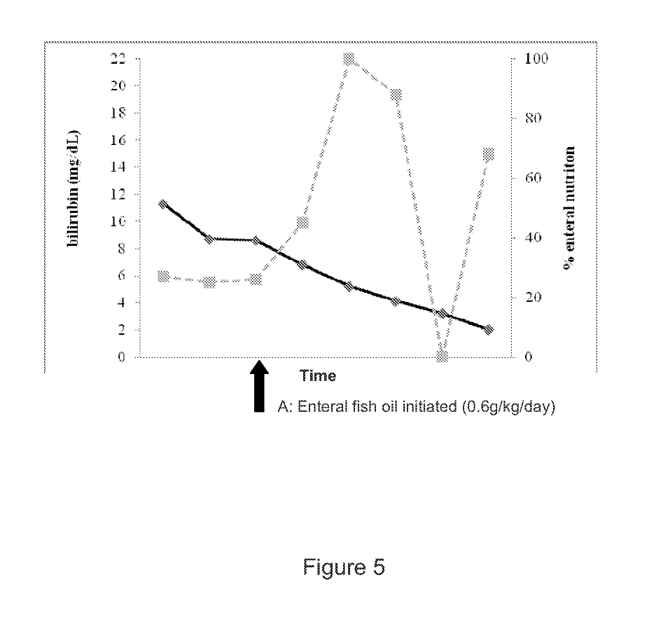

A female born at 27 weeks gestation developed NEC at one week of age requiring a small bowel resection and jejunostomy placement. Patient 5 was left with approximately 32 cm of small bowel with an intact ileocecal valve and three ostomies. Postoperatively patient 5 was maintained on PN with slow advancement of EN. By eight weeks of age she had developed PNALD. Patient 5 underwent reanastamosis approximately three months after her small bowel resection. At seven months of age, EN was still slowly being advanced and bilirubin remained increased. Enteral fish oil was initiated at 0.6 g/kg/day. Bilirubin decreased, enteral feeding tolerance improved and six weeks after initiation of enteral fish oil, PNALD had completely reversed (see FIG. 5).

### Example 6

### Case Report: Patient 6

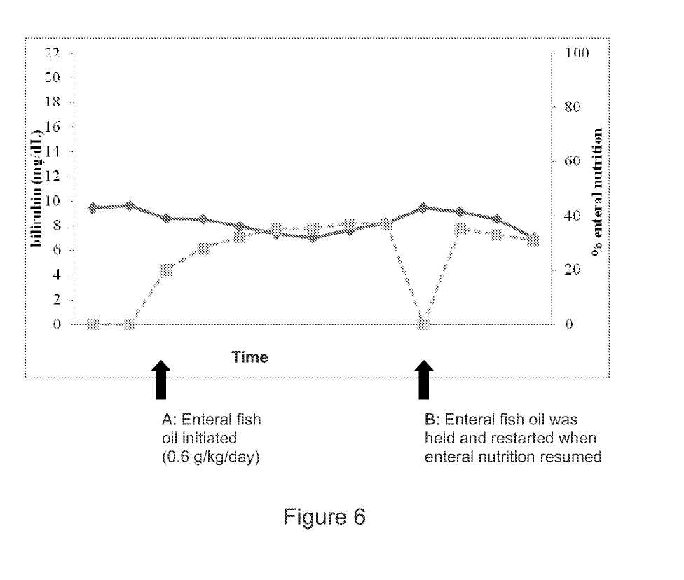

A female born at 26 weeks gestation developed NEC at four weeks of age and was transferred to Le Bonheur Children's Medical Center for surgical management. Upon arrival, patient 6 had already developed PNALD. NEC was medically managed for one week but disease progression required surgical intervention. The bowel was found to be ischemic and friable with multiple perforations requiring extensive resections including the loss of the ileocecal valve. A jejunocolostomy was made leaving approximately eight centimeters of small bowel which was reanastamosed three months later. Postoperatively patient 6 was maintained on PN and low volume trophic EN was initiated. At six months of age, patient 6 was receiving PN with minimal EN and bilirubin remained increased at 9.6 mg/dL. At this time enteral fish oil was started (0.6 g/kg/day) and bilirubin decreased; however, patient 6 continued to experience feeding intolerance. After six weeks of enteral fish oil the bilirubin decreased to 7 mg/dL. Patient 6 then developed bloody stools which required EN and enteral fish oil to be stopped for 2 weeks for medical management of NEC, including NPO status and IV antibiotics. Her INR had not changed from values prior to starting enteral fish oil. During the two weeks that patient 6 was not receiving EN or fish oil, bilirubin increased to 9.4 mg/dL. When EN was restarted, fish oil was also restarted and bilirubin again decreased. Six months after initiation of enteral fish oil, the patient was still on PN with supplemental EN and bilirubin was approximately 7 mg/dL. She was discharged on a combination of PN, EN, and enteral fish oil. Patient 6 has had multiple readmissions to the hospital and there has been another occasion where enteral fish oil was temporarily discontinued. Every time enteral fish oil has been stopped bilirubin has increased then decreased upon re-initiation. To date, her bilirubin remains approximately 7 mg/dL (see FIG. 6).

### Discussion of Examples 1-6

A notable improvement in PNALD and EN advancement was associated with enteral fish oil supplementation in four of the six patients. The establishment of enteral feedings is generally thought to be a significant factor in the prevention and treatment of PNALD. Horslen et al. reported improved enteral tolerance in infants with short bowel syndrome and end stage liver disease after liver transplantation (Horslen et al., 2002). This suggests that the resolution of liver disease, whether it occurs via transplantation or medical management, might have a positive impact on enteral tolerance. In contrast, Javid et al. retrospectively evaluated 12 infants with PNALD and observed reversal of PNALD in only two patients on PN while enteral feeds were advancing. PNALD did not reverse in the other 10 patients until approximately four months after PN was discontinued and patients were receiving full EN (Javid et al., 2005). While some preliminary case reports and studies of the use of fish oil based IVFE for the treatment of PNALD have shown promise, enteral administration of omega-3 fatty acid compositions, e.g. fish oil, has not been considered for treating PNALD prior to the instant disclosure. Surprisingly, the results of the studies described herein demonstrate that enteral fish oil administration provided for the dramatic improvement in PNALD and enteral feeding tolerance, and in some cases complete reversal of PNALD.

Previous reports of fish oil based IVFE have utilized a dose of 1 g/kg/day as the exclusive source of fats (Gura et al., 2006; Gura et al., 2008). Patients in the instant studies were started on a dose of 1 g/kg/day. The dramatic improvement of PNALD observed in patient 1 with a total dose of 0.15 g/kg/day suggests that doses much lower than what has been previously reported with intravenous fat emulsions (IVFE) approaches.

As compared to patients with PNALD that did not receive fish oil, these six patients had better overall outcomes which are believed to be attributed to the administration of enteral fish oil. In the four patients that had reversal of PNALD, this occurred while receiving some PN, whereas typically PNALD does not reverse until several weeks after the patient has been weaned off of PN.

In conclusion, PNALD remains a potentially devastating disease with limited treatment options. Providing appropriate macro- and micro-nutrients parenterally and use of EN as early as possible remain aspects of managing PNALD. While fish oil based IVFE has shown promise for treating PNALD, enteral administration of omega-3 fatty acid compositions, e.g. fish oil, has not been evaluated prior to the instant disclosure, presumably due to the assumption of enteral intolerance in subjects receiving PN. As demonstrated herein, enteral supplementation of omega-3 fatty acid compositions, e.g. fish oil, is a surprisingly safe and effective adjunctive treatment for PNALD.

### Example 7

### Omega-3 Fatty Acid Compositions

Liquid omega-3 products are formulated from a marine sourced oil, de-odorized marine or algal source raw material. Alternatively, a micro-encapsulated powdered preparation is used as a starting material and formulated into either a suspension or emulsion product. The provided compositions can be a concentrated source of omega-3 polyunsaturated fatty acids that is outside the range of what is normally supplemented in neonatal or infant formulas. They can be formulated to provide the appropriate dosages of DHA and EPA to meet a therapeutic endpoint of resolving or preventing PNALD in a convenient to use volume.

Also provided are flavored, e.g. citrus, suspensions or emulsions. Using a variety of commercially available flavoring agents a formulation with acceptable sensory components (smell, taste, texture) is achieved. Different flavors such as citrus, cherry, raspberry, and tangerine (or other flavors) are evaluated for a representative omega-3 fatty acid composition. While some product delivery can be via a feeding tube, developing a de-odorized liquid formulation can make oral, i.e. enteral, drug delivery safer, easier, and more palatable to both patient and care giver.

The omega-3 fatty acid composition can comprise an emulsion or suspension of omega-3 fatty acids using commercially available sources of DHA and EPA as either the triglyceride or as the ethyl ester form. These commercial forms are available from several companies and are sourced from either fish or algal materials. In some embodiments, omega-3 fatty acid compositions of the presently disclosed subject matter are in the ethyl ester form, or substantially in the ethyl ester form. In some embodiments, omega-3 fatty acid compositions of the presently disclosed subject matter are in the triglyceride form, or substantially in the triglyceride form. In some embodiments, algal sourced omega-3 fatty acid compositions of the presently disclosed subject matter are substantially in the triglyceride form. In some embodiments, algal sourced omega-3 fatty acid compositions of the presently disclosed subject matter are substantially in the triglyceride form, wherein all three positions are DHA for most of the oil (some diglyceride). In some embodiments, algal sourced omega-3 fatty acid compositions of the presently disclosed subject matter are substantially in the triglyceride form, wherein the composition is 35% DHA (wt/wt) with little to no EPA.

After providing a stable emulsion/suspension of omega-3 that is suitable for oral or feeding tube administration, appropriate flavoring and masking agents can be added to produce a pharmaceutically elegant product, e.g. a product with enhanced taste and smell.

A particular dosage of DHA and EPA shown to be effective (see, e.g. Examples 1-6) can be incorporated into both an oil-in-water emulsion and a powder-in-liquid suspension. A variety of commercially available omega-3 raw materials can be employed to provide a formulation that closely matches an effective dosage of DHA and EPA in an acceptable volume. In some cases two or three sources of oil and of powder are employed.

A variety of masking agents and flavorings are used to develop a series of formulations for sensory evaluation. These formulations are evaluated by experienced pediatric care-givers for acceptable smell, flavor, and texture.

Formulations that have acceptable sensory properties can be provided to achieve products as either an emulsion (oil in water) and as a suspension. Accelerated storage at 30° C. and 40° C. can be used to test preliminary stability. Acceptable formulations can be judged by sensory evaluation, appearance and chemical stability including evaluation of the fatty acid profile by gas chromatography.

The resulting product can be an acceptable flavored emulsion or suspension of omega-3 fatty acids (DHA and EPA) to allow for easier administration to children or adults. The resulting product can also be easier to administer versus drawing up contents out of a capsule with a needle. This formulation can also allow for a more accurate and consistent dosage of omega-3 fatty acids.

### Materials and Methods Used in the Examples 8-10

Emulsions were achieved by placing approximately 250 mL of water in a 600 mL beaker and heating to 50 to 60° C. while stirring. Sugar was dissolved in the water. Similarly, approximately 250 mL of oil was heated in a 500 mL beaker to 50 to 60° C. while stirring. Soy lecithin and carrageenan (if used) were dissolved slowly in the oil. A POLYTRON™ homogenizer was positioned in the water to achieve efficient mixing. The oil mixture was then slowly added to the water while maintaining the temperature at 50 to 60° C. The speed of mixing was increased as necessary (as the emulsion forms and viscosity increases) to achieve a complete mixture.

After completing the oil addition, the homogenizer was adjusted to a medium speed and mixing was continued for at least 30 minutes or as necessary to achieve a stable emulsion. Flavoring and masking agents (if used) were added to the completed emulsion and mixed for an additional five to ten minutes. Alternatively, flavoring and masking agents can be dissolved in the water or oil (depending on solubility) prior to mixing.

### Example 8

### Omega-3 Fatty Acid Composition Formulated from a Marine Sourced Oil

An embodiment of the presently disclosed subject matter is a 50:50 (vol/vol) oil-in-water emulsion in which the oil is a fish oil mixture of EPA and DHA (Fish Oil E3322, Bioriginal Food and Science Corp., Saskatoon, Saskatchewan, Canada, or equivalent). In some embodiments, this composition can also contain 15% (wt/vol) sugar, 1.5% (wt/vol) soy lecithin (Solec F, Deoiled Soy Lecithin, The Solae Company, St. Louis, Mo., United States of America, or equivalent), with or without 0.5% carrageenan (Viscarin PC 389, FMC BioPolymer, Philadelphia, Pa., United States of America, or equivalent) and 1.5% (vol/vol) flavoring (Tropical Punch, Gold Coast Corp., Commerce, Calif., United States of America, or equivalent) and 1.0% masking agent (Masking Flavor, NAT, 936.0625U, FONA Int., Inc., Geneva, Ill., United States of America, or equivalent). Emulsion was achieved with a homogenizer (POLYTRON™ Homogenizer, Model PT10/35, Brinkman Instruments, Inc., Westbury, N.Y., United States of America, or equivalent) initially set on low and progressively increased in speed to achieve a complete mixture of oil-in-water. After reaching a complete mixture visually, the homogenizer speed was set to approximately 50% of maximum and was continued for 30 minutes. The resulting emulsion is stable at room temperature and at 4-5° C. for at least 30 days.

### Example 9

### Omega-3 Fatty Acid Composition Formulated from a De-Odorized Marine Sourced Oil

An embodiment of the presently disclosed subject matter is a 50:50 (vol/vol) oil-in-water emulsion in which the oil is a steam de-odorized fish oil mixture of EPA and DHA (MEG-3™ EPA3E1615 Oil, Ocean Nutrition, Dartmouth, Nova Scotia, Canada, or equivalent). This embodiment also contains 12.5% (wt/vol) sugar, 1.5% (wt/vol) soy lecithin (Solec F, Deoiled Soy Lecithin, The Solae Company, St. Louis, Mo., United States of America, or equivalent), and 1.0% (vol/vol) flavoring (Citrus Punch, Va. Dare Co., Brooklyn, N.Y., United States of America, or equivalent). Emulsion was achieved with a homogenizer (POLYTRON™ Homogenizer, Model PT10/35, Brinkman Instruments, Inc., Westbury, N.Y., United States of America, or equivalent) initially set on low and progressively increased in speed to achieve a complete mixture of oil-in-water. After reaching a complete mixture visually, the homogenizer speed was set to approximately 50% of maximum and was continued for 30 minutes. The resulting emulsion is stable at room temperature and at 4-5° C. for at least 30 days.

### Example 10

### Omega-3 Fatty Acid Composition Formulated from an Algal Source

Another embodiment of the presently disclosed subject matter is a 40:60 (vol/vol) oil-in-water emulsion in which the oil is an algal oil mixture of EPA and DHA (Rosemary Free Algal Vegetable Oil DHA-S, Martek Biosciences corp., Columbia, Md., United States of America, or equivalent). This embodiment also contains 12.5% (wt/vol) sugar, 1.5% (wt/vol) soy lecithin (Solec F, Deoiled Soy Lecithin, The Solae Company St. Louis, Mo., United States of America, or equivalent), with or without 0.5% carrageenan (Viscarin PC 389, FMC BioPolymer, Philadelphia, Pa., United States of America, or equivalent) and 1.0% (vol/vol) flavoring (Tropical Punch, Gold Coast Corp., Commerce, Calif., United

States of America., or equivalent). Emulsion was achieved with a homogenizer (POLYTRON™ Homogenizer, Model PT10/35, Brinkman Instruments, Inc., Westbury, N.Y., United States of America, or equivalent) initially set on low and progressively increased in speed to achieve a complete mixture of oil-in-water. After reaching a complete mixture visually, the homogenizer speed was set to approximately 50% of maximum and was continued for 30 minutes. The resulting emulsion is stable at room temperature and at 4-5° C. for at least 30 days.

### Example 11

### Effects of EPA and DHA on Hepatocellular Apoptosis Induced by the Hydrophobic Bile Acid

Although not intended to be limited by any particular theory or mechanism of action, one possible mechanism of action responsible for the notable improvement in PNALD and EN advancement in subjects administered omega-3 fatty acid compositions is the attenuation of apoptosis induced by high levels of retained hydrophobic bile acids. As such, a study was conducted to determine the effects of EPA and DHA on hepatocellular apoptosis induced by the hydrophobic bile acid, chenodeoxycholic acid (CDCA).

Cultured HepG2 cells were treated with 50, 100, or 200 μM CDCA in the presence and absence of 10 μM EPA, 10 μM DHA, or 5 μM EPA+5 μM DHA. Controls included cells incubated with vehicle alone (EtOH). Apoptosis was evaluated after 4, 8, 12, 18, and 24 hours using the Apo-ONE®

Homogeneous Caspase-3/7 Assay (Promega Corporation, Madison, Wis., United States of America). Specific apoptotic mediators (Fas and TRAIL-R2) were evaluated at 0.5, 1, 1.5, 2, and 4 hrs using quantitative real-time RT-PCR.

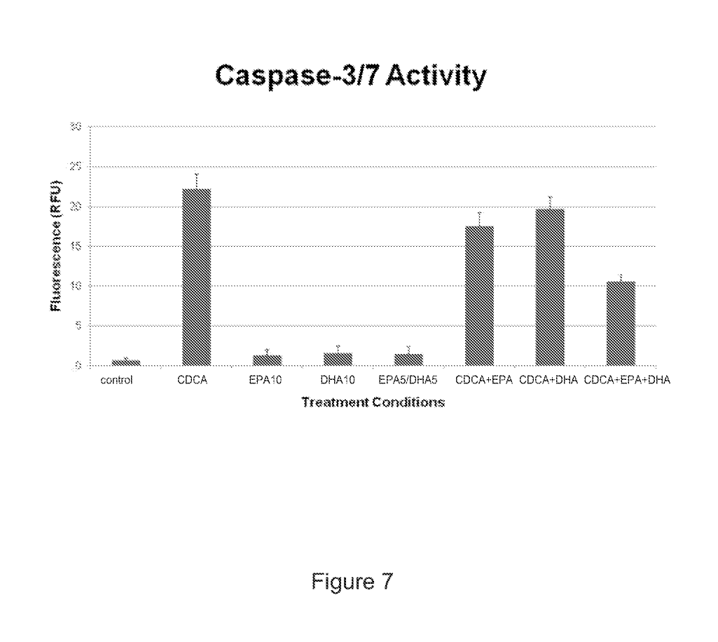

Treatment of HepG2 cells with 200 μM CDCA resulted in peak caspase activity at 12 hours. Treatment with EPA alone and DHA alone resulted in 22% and 9% caspase-3/7 attenuation, respectively. Caspase-3/7 activity was attenuated by 52% when cells were treated with a combination of EPA and DHA (p=0.0034) (FIG. 7). Peak Fas and TRAIL-R2 mRNA expression was observed at 0.5 hours. There was a 4.7-fold increase in Fas mRNA levels when cells were incubated with CDCA 200 μM alone, as compared to no increase in Fas mRNA levels when incubated with CDCA 200 μM with the addition of EPA 1 μM, EPA 10 μM, or 5 μM EPA+5 μM DHA (p<0.01). There was a 2-fold increase in TRAIL-R2 mRNA levels when cells were incubated with CDCA 100 μM and 200 μM alone, as compared to no increase in TRAIL-R2 mRNA levels when incubated with both CDCA and EPA 1 μM, EPA 10 μM, or 5 μM EPA+5 μM DHA (p<0.01). No significant difference between treatment with EPA alone, DHA alone, or combination of EPA/DHA was observed in Fas or TRAIL-R2 mRNA expression.

The combination of EPA and DHA resulted in a synergistic attenuation of bile acid-induced hepatocellular apoptosis evaluated by caspase 3/7 activity, as compared to treatment with EPA and DHA separately, in HepG2 cells. The combination of EPA and DHA did not result in a synergistic attenuation of the up-regulation of Fas or TRAIL-R2. These data suggest that EPA and DHA can attenuate bile acid-induced apoptosis.

### Example 12

### Effects of EPA and DHA on Fas and TRAIL-R2 mRNA Expression and Hepatocellular Apoptosis Induced by Hydrophobic Bile Acid

Material and Methods

Cell culture. HepG2 cells were obtained from the American Type Culture Collection (Rockville, Md., United States of America) and cultured in EMEM supplemented with 10% FBS, 50 U/ml penicillin, and 37.5 U/ml streptomycin (growth medium). Cells were incubated at 37° C. with 5% CO₂ in a humidified incubator. Passages 25-45 were used for these experiments.

Experimental Design. HepG2 cells were plated and grown to 95% confluence for all experiments, except for fluorescent cell staining, where cells where grown to 50% confluence. Cells were treated with CDCA 50-200 mM (Sigma-Aldrich), ±EPA 1-10 μM (Nu-check Prep, Elysian, Minn., United States of America), and ±DHA 10 μM (Nu-check Prep). HepG2 cells were treated for 4, 8, 12, 18, 24 hours for cell viability and caspase assays. Cells were treated for 0.5, 1, 1.5, 2, 4 hour for mRNA analysis by quantitative real time RT-PCR.

Cell Viability. Cell viability was evaluated after treating cells, as described above, followed by trypsinization in order to disperse cells into a 0.2% trypan blue (Sigma-Aldrich) cell suspension mixture. After staining, 10 μL of cell suspension was placed on a hemocytometer with a glass cover slip and evaluated using an inverted microscope (25× magnification). All cells were counted and viability was accessed.

Nuclear staining. Cells were grown on glass inserts and once 50% confluence was achieved, cells were treated with CDCA±EPA for 2 hours. Immediately following treatment, cells were washed with phosphate buffered saline (PBS), fixed with methanol (−10° C.) for 15 minutes, then washed with PBS. Fixed cells were then stained for 5 minutes with ethidium bromide (100 μg/ml) and acridine orange (100 μg/ml) (EB/AO) dye mixture (Sigma-Aldrich) in PBS. Following staining, cells underwent 5 washes prior to being mounted on glass slides using VECTASHIELD® mounting medium and analyzed using an Olympus America Inc., IX50-FLA inverted reflected light fluorescence microscope (Lake Success, N.Y., United States of America).

Caspase assay. Apoptosis was evaluated using the Apo-ONE® Homogeneous Caspase-3/7 Assay purchased from Promega Corporation (Madison, Wis., United States of America) and performed according to the manufacturer's instructions. HepG2 cells were treated for 4, 8, 12, 18, 24 hours followed by the addition of caspase-3/7 reagent and incubation for 4 hours in the dark on a rocking shaker at low speed. Results were read at fluorescein 485 nm/535 nm with a Victor 2, Perkin-Elmer Wallace 1420 multilabel counter (Shelton, Conn., United States of America).

Quantitative RT-PCR. Total RNA was isolated from confluent cultures according to manufacturer's instructions with TRIzol™ reagent (Invitrogen, Carlsbad, Calif., United States of America). First-strand cDNAs were synthesized from 2 μg of total RNA in a 21 μl reaction volume using the SuperScript™ First-Strand Synthesis System for RT-PCR (Invitrogen) in accordance with the manufacturer's instructions. Quantitative PCR was performed in triplicate using the Model 7500 Sequence Detection System (Applied Biosystems, Foster City, Calif., United States of America). Independent PCRs were performed using the same cDNA for both the gene of interest and the 18S rRNA, using the SYBR Green PCR Master Mix (Applied Biosystems). Specific primers were designed for the genes of interest (Fas, TRAIL-R2, and IL-6). A dissociation curve was generated at the end of each PCR cycle to verify that a single product was amplified using software provided with the 7500 Sequence Detection System. The change in fluorescence of SYBR Green in every cycle was monitored by the system software, and the threshold cycle (C_{T}) above the background for each reaction was calculated. The C_{T} value of 18S rRNA was subtracted from that of the gene of interest to obtain a ΔC_{T} value. The ΔC_{T} value of an arbitrary calibrator (e.g., sample treated with ethanol as a vehicle control) was subtracted from the ΔC_{T} value of each sample to obtain a ΔΔC_{T} value. The mRNA expression level relative to the calibrator was expressed as 2^{-ΔΔCT}.

Statistical analysis. All data represent at least three separate and independent experiments. Data are provided as mean±SEM. A one-way ANOVA was used to compare differences between groups and a post-hoc Tukey's LSD test was used to correct for multiple comparisons. A single-tailed P value of 0.05 was used to reject the null hypothesis.

Results

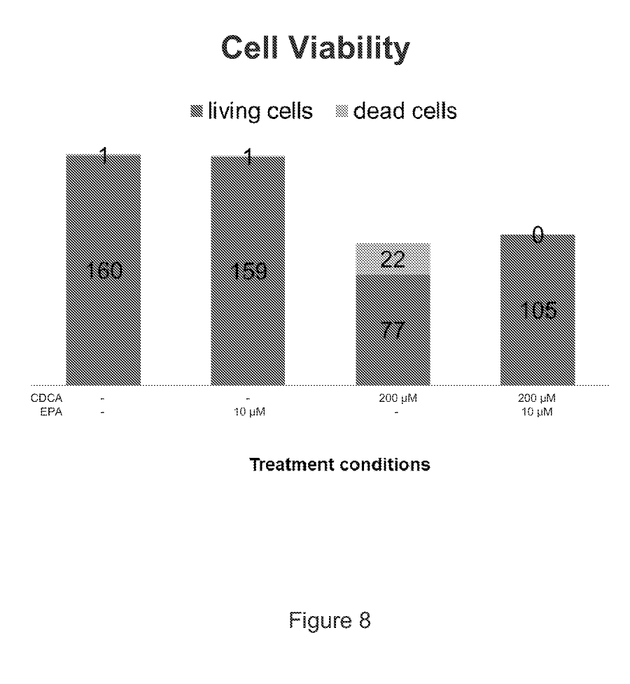

Cell Viability. After a 2 hour treatment with CDCA, 88% of cells were viable, whereas 100% of cells were viable after treatment with CDCA and EPA (FIG. 8). Although cell viability was 100% in cells treated with CDCA and EPA, the total cell count was only 75% of cells exposed to medium alone. Cells treated with EPA alone resulted in similar viability to control and vehicle control: whereas, cells treated with CDCA alone had both a decrease total number and increased number of dead cells. When cells were treated with CDCA+EPA the cell number was similar, but there were no dead cells

Nuclear Staining. Cells treated with EPA alone appear similar to control cells with all green viable cells. When cells were treated with CDCA alone, not only is there free chromatin, which is stained orange, but cellular integrity is lost. Cells treated with CDCA+EPA exhibited some early stage apoptosis shown by orange staining, but cellular integrity was maintained.

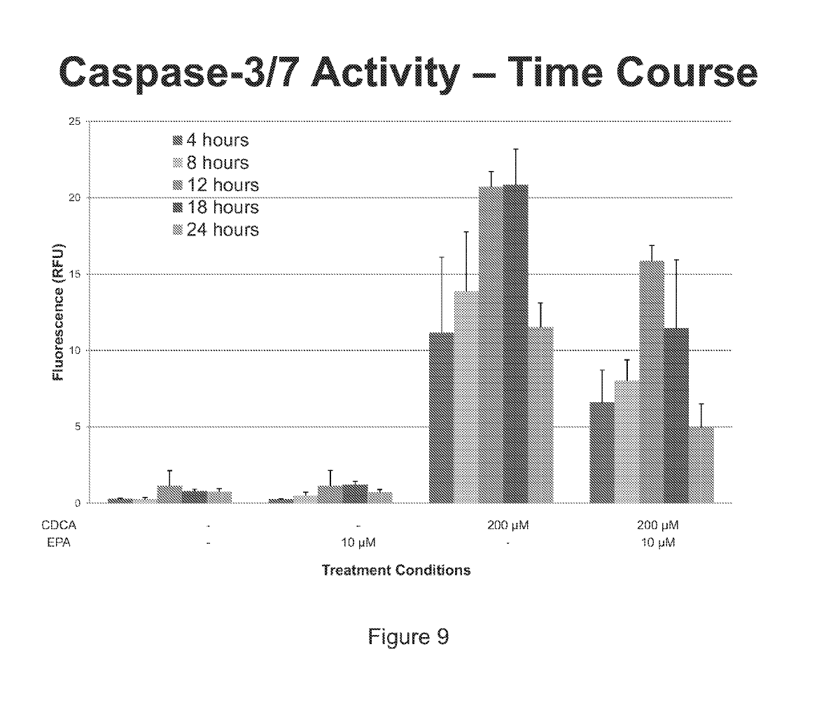

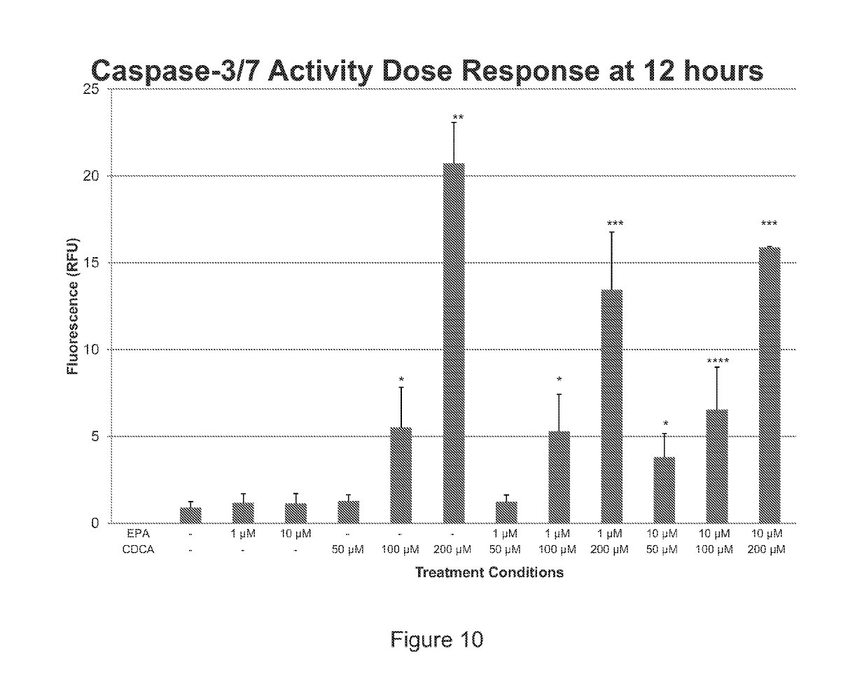

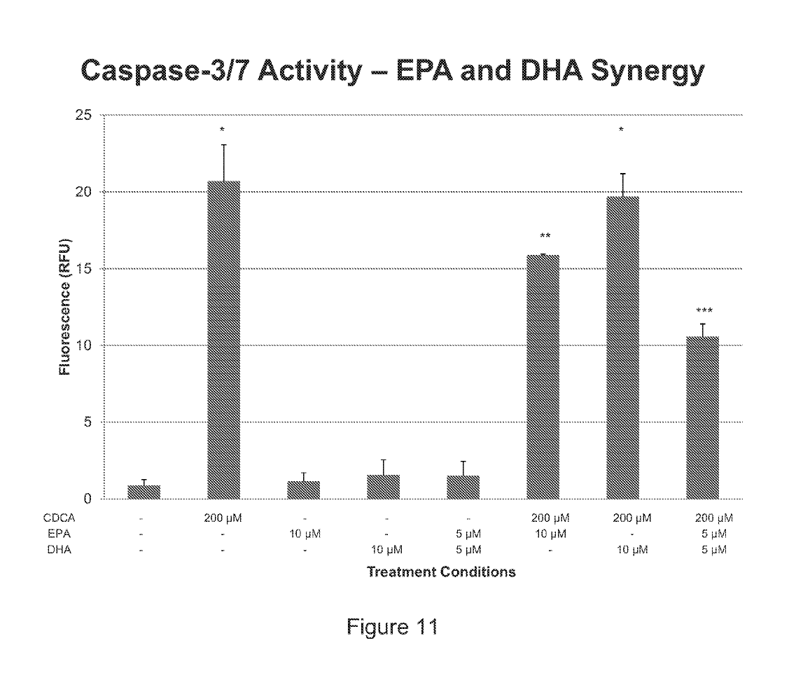

Caspase 3/7 Activity. Caspase activity was evaluated at 4, 8, 12, 18, and 24 hours (FIG. 9). Activity peaked between 12 and 18 hours, therefore, a 12 hour time point was chosen for data reporting. A dose-dependent apoptotic effect of CDCA was observed across all time points, and a significant attenuation effect was observed with 1 and 10 μM EPA with treatment with 200 μM CDCA (FIG. 10). Peak caspase-3/7 activity was observed with CDCA 200 μM. Treatment with EPA alone and DHA alone resulted in 22% and 9% caspase-3/7 attenuation, respectively (FIG. 3c). Caspase-3/7 activity was attenuated by 52% when treated with a combination of EPA and DHA (p=0.0034) (FIG. 11).

Fas and TRAIL-R2 mRNA Levels. Peak Fas and TRAIL-R2 mRNA expression was observed at 0.5 hours (FIGS. 12 and 13). There was a 4.7-fold increase in Fas mRNA levels when cells were incubated with 200 μM CDCA alone, as compared to no increase in Fas mRNA levels when incubated with CDCA 200 μM with the addition of EPA 1 μM, EPA 10 μM, or 5 μM EPA+5 μM DHA (p<0.01) (FIG. 12). There was a 2-fold increase in TRAIL-R2 mRNA levels when cells were incubated with CDCA 100 μM and 200 μM alone, as compared to no increase in TRAIL-R2 mRNA levels when incubated with both CDCA and EPA 1 μM, EPA 10 μM, or 5 μM EPA+5 μM DHA (p<0.01) (FIG. 13). No significant difference between treatment with EPA alone, DHA alone, or combination of EPA/DHA was observed in Fas or TRAIL-R2 mRNA expression. Interesting, EPA and DHA separately appeared to induce a low level of both Fas and TRAIL-R2 mRNA when no CDCA was present. However, this effect was not observed with the combination of the two fatty acids with or without CDCA (FIGS. 12 and 13).

Discussion. The results from this study show that the omega-3 fatty acids EPA and DHA significantly attenuate CDCA-induced apoptosis in cultured hepatocytes. The results demonstrate: (1) CDCA dose-dependent induction of apoptosis documented by caspase 3/7 activity and Fas and TRAIL-R2 mRNA expression; (2) attenuation of CDCA-induced apoptosis via caspase 3/7 by EPA; (3) synergistic attenuation of CDCA-induced apoptosis by the combination of EPA and DHA as documented by caspase-3/7 activity.

HepG2 cells treated with CDCA for 0.5 hour exhibited increased expression of both Fas and TRAIL-R2 mRNA in a dose-dependent manner. This up-regulation of expression of Fas and TRAIL-R2 mRNA with CDCA treatment was attenuated when cells were treated with EPA, DHA, or a combination of both EPA and DHA. A synergistic attenuation of CDCA-induced apoptosis via caspase-3/7 with a combination of EPA and DHA was observed, yet Fas and TRAIL-R2 mRNA expression was not significantly different from that observed with EPA alone, DHA alone, or a 1:1 molar ratio of EPA to DHA. This discrepancy may suggest that DHA and EPA are involved in multiple pathways of apoptosis attenuation. It is noted that EPA and DHA are structurally different and have been shown to have varying effects on cellular functions. For example, the additional double bond in DHA compared to EPA creates an increased number of bends in the acyl chain that cause shortening. Length and saturation of hydrocarbon chains can greatly affect mitochondrial membrane permeability (Wojtczak et al., 1999). As discussed previously herein, hepatocyte apoptosis is mediated via the mitochondria, and changes in mitochondrial membrane permeability could influence hepatocellular apoptosis. The EPA and DHA synergy in attenuation of CDCA-induced apoptosis observed may represent the end-result of omega-3 fatty acid functioning via multiple mechanisms, including alteration of cell membrane fluidity, receptor binding, transcriptional regulation, and non-transcriptional regulation of RNA levels resulting in the anti-apoptotic effects.

### Example 13

### Effect of EPA on Pro-Inflammatory Cytokine (IL-6) mRNA Expression in HepG2 Cells

A study was conducted to determine the effect of EPA on pro-inflammatory cytokine (IL-6) mRNA expression in HepG2 cells incubated with chenodeoxycholic acid (CDCA). Cultured HepG2 cells were treated with EPA (10 uM), CDCA (200 uM) or CDCA (200 uM) and EPA (10 uM).

IL6 mRNA levels were measured by quantitative RT-PCR after a 2 hour incubation in a HepG2 cells. Values are based on a fold change relative to the vehicle control. Statistical significance was determined using an ANOVA with Tukey's LSD. Each bar represents mean±SEM for data from three culture wells. Statistical significance was determined using an ANOVA with Tukey's LSD. Treatment conditions represented with different symbols above the bars are significantly different at p<0.05. Those with the same or no symbols are not significantly different. See FIG. 14.

Treatment with CDCA significantly increased pro-inflammatory cytokine (IL-6) mRNA expression in HepG2 cells. Treatment with EPA attenuated the increase in pro-inflammatory cytokine (IL-6) mRNA expression caused by CDCA. These data suggest that omega-3 fatty acids such as EPA and DHA can treat or prevent diseases with an inflammatory component.

### REFERENCES

1. Merritt R. J., 1986, Cholestasis associated with total parenteral nutrition, J Pediatr Gastroenterol Nutr 5:9-22.
2. Beale E F, Nelson R M, Bucciarelli R L, Donnelly W H, Eitzman D V, 1979, Intrahepatic cholestasis associated with parenteral nutrition in premature infants, Pediatrics 64:342-47.
3. Nakai H, Landing B H, 1961, Factors in the genesis of bile stasis in infancy Pediatrics 27:300-7.
4. Gura K M, Duggan C P, Collier S B, Jennings R W, Folkman J, Bistrian B R, Puder M, 2006, Reversal of parenteral nutrition-associated liver disease in two infants with short bowel syndrome using parenteral fish oil: implications for future management, Pediatrics 118:197-201.
5. Gura K M, Lee S, Valim C, et al., 2008, Safety and efficacy of a fish oil-based fat emulsion in the treatment of parenteral nutrition-associated liver disease, Pediatrics 121:678-686.
6. De Marcho G, Sordion D, Bruzzese E, Di Caro S, Mambretti D, Tramontano A, et al., 2006, Early treatment with ursodeoxycholic acid for cholestasis in children on parenteral nutrition because of primary intestinal failure, Aliment Pharmacol Ther 24:387-394.
7. Horslen S P, Sudan D L, lyer K R, 2002, Isolated liver disease in infants with end-stage liver disease associated with short bowel syndrome, Ann Surg 235:435-439.
8. Javid P J, Collier S, Richardson D, et al., 2005, The role of enteral nutrition in the reversal of parenteral nutrition-associated liver dysfunction in infants, J Pediatr Sur 40:1015-18.
9. U.S. Pat. No. 4,491,589
10. Shimizu T, Fujii T, Suzuki R, et al. Effects of highly purified eicosapentaenoic acid on erythrocyte fatty acid composition and leukocyte and colonic mucosa leukotriene B4 production in children with ulcerative colitis. J Pediatr Gastroenterol Nutr 2003; 37:581-5.
11. Panchaud A, Sauty A, Kernen Y, et al. Biological effects of a dietary omega-3 polyunsaturated fatty acids supplementation in cystic fibrosis patients: a randomized, crossover placebo-controlled trial. Clin Nutr 2006; 25:418-27.
12. Pontes-Arruda A, Aragao A M, Albuquerque J D. Effects of enteral feeding with eicosapentaenoic acid, gamma-linolenic acid, and antioxidants in mechanically ventilated patients with severe sepsis and septic shock. Crit Care Med 2006; 34:2325-33.
13. Yang R, Harris W S, Vernon K, et al. Prefeeding with omega-3 fatty acids suppresses inflammation following hemorrhagic shock. JPEN J Parenter Enteral Nutr; 2010, 34:496-502.
14.Skulas-Ray A C, Kris-Etherton P M, Harris W S, Vanden Heuvel J P, Wagner P R, West S G. Dose-response effects of omega-3 fatty acids on triglycerides, inflammation, and endothelial function in healthy persons with moderate hypertriglyceridemia. Am J Clin Nutr., 2010; Epub ahead of print, Dec. 15, 2010.
15. Noel S E, Newby P K, Ordovas J M, Tucker K L. Adherence to an (n-3) fatty acid/fish intake pattern is inversely associated with metabolic syndrome among Puerto Rican adults in the Greater Boston area. J Nutr; 2010, 140:1846-54.
16. Murff H J, Shu X O, Li H, et al. Dietary polyunsaturated fatty acids and breast cancer risk in Chinese women: A prospective cohort study. Int J Cancer., 2010 Epub ahead of print.
17. Clayton E H, Hanstock T L, Hirneth S J, Kable C J, Garg M L, Hazell P L. Long-chain omega-3 polyunsaturated fatty acids in the blood of children and adolescents with juvenile bipolar disorder. Lipids 2008; 43:1031-8.
18. Burrows T, Collins C E, Garg M L. Omega-3 index, obesity and insulin resistance in children. Int J Pediatr Obes., 2011, Epub ahead of print.
19. Higuchi H, Gores G J. Bile acid regulation of hepatic physiology: IV. Bile acids and death receptors. Am J Physiol Gastrointest Liver Physiol. May 2003; 284(5):G734-738.
20. Amaral J D, Castro R E, Sola S, Steer C J, Rodrigues C M. p53 is a key molecular target of ursodeoxycholic acid in regulating apoptosis. J Biol Chem. Nov. 23, 2007; 282(47):34250-34259.
21. Yang J I, Yoon J H, Myung S J, et al. Bile acid-induced TGR5-dependent c-Jun-N terminal kinase activation leads to enhanced caspase 8 activation in hepatocytes. Biochem Biophys Res Commun. Sep. 14, 2007; 361(1):156-161.
22. Reinehr R, Becker S, Wettstein M, Haussinger D. Involvement of the Src family kinase yes in bile salt-induced apoptosis. Gastroenterology. November 2004; 127(5):1540-1557.
23. Gumpricht E, Dahl R, Devereaux M W, Sokol R J. Licorice compounds glycyrrhizin and 18beta-glycyrrhetinic acid are potent modulators of bile acid-induced cytotoxicity in rat hepatocytes. J Biol Chem. Mar. 18, 2005; 280(11):10556-10563.
24. Bernt C, Vennegeerts T, Beuers U, Rust C. The human transcription factor AP-1 is a mediator of bile acid-induced liver cell apoptosis. Biochem Biophys Res Commun. Feb. 17, 2006; 340(3):800-806.
25. Park S E, Lee S W, Hossain M A, et al. A chenodeoxycholic derivative, HS-1200, induces apoptosis and cell cycle modulation via Egr-1 gene expression control on human hepatoma cells. Cancer Left. Oct. 18, 2008; 270(1):77-86.
26. Higuchi H, Bronk S F, Takikawa Y, et al. The bile acid glycochenodeoxycholate induces trail-receptor 2/DR5 expression and apoptosis. J Biol Chem. Oct. 19, 2001; 276(42):38610-38618.
27. Wojtczak L, Wieckowski M R. The mechanisms of fatty acid-induced proton permeability of the inner mitochondrial membrane. J Bioenerg Biomembr. October 1999; 31(5):447-455.

It will be understood that various details of the presently disclosed subject matter may be changed without departing from the scope of the presently disclosed subject matter. Furthermore, the foregoing description is for the purpose of illustration only, and not for the purpose of limitation.

1. A method of treating parenteral nutrition associated liver disease (PNALD) in a subject, the method comprising: providing a subject with PNALD; and administering to the subject an effective amount of an omega-3 fatty acid composition, wherein the omega-3 fatty acid composition is administered enterally.

2. The method of claim 1, wherein the subject is an infant having a low birth weight, very low birth weight, extremely low birth weight, a low gestational age, short bowel syndrome (SBS), necrotizing entercolitis (NEC), gastroeschesis, omphelacele, atresias, Hirschprungs disease, functional short bowel syndrome or a combination thereof.

3. The method of claim 1, wherein a subject with PNALD is a subject having a direct bilirubin concentration of >2 mg/dL and/or elevated transaminases, GGT, alk phos, or clinical correlation.

4. The method of claim 1, wherein the omega-3 fatty acid composition comprises docosahexanoic acid (DHA) and eicosapentaenoic acid (EPA).

5. The method of claim 1, wherein the omega-3 fatty acid composition comprises fish oil or deodorized fish oil.

6. The method of claim 1, wherein the omega-3 fatty acid composition comprises algal sourced omega-3 fatty acids.

7. The method of claim 6, wherein the algal sourced omega-3 fatty acids are in the triglyceride or ethyl ester form.

8. The method of claim 1, wherein the subject is receiving parenteral nutrition (PN).

9. The method of claim 1, wherein the enteral administration comprises oral administration.

10. The method of claim 1, wherein treating comprises an enhanced ability for the subject to tolerate enteral feeding as compared to a subject receiving PN but not administered an effective amount of an omega-3 fatty acid composition.

11. A method of preventing PNALD in a subject, the method comprising: providing a subject at risk for developing PNALD; and administering to the subject an effective amount of an omega-3 fatty acid composition, wherein the omega-3 fatty acid composition is administered enterally.

12. The method of claim 11, wherein the subject at risk for developing PNALD is an infant receiving PN and having a low birth weight, very low birth weight, extremely low birth weight, a low gestational age, SBS, NEC, gastroeschesis, omphelacele, atresias, Hirschprungs disease, functional short bowel syndrome or a combination thereof.

13. The method of claim 11, wherein the omega-3 fatty acid composition comprises DHA and EPA.

14. The method of claim 11, wherein the omega-3 fatty acid composition comprises fish oil or deodorized fish oil.

15. The method of claim 11, wherein the omega-3 fatty acid composition comprises algal sourced omega-3 fatty acids.

16. The method of claim 15, wherein the algal sourced omega-3 fatty acids are in the triglyceride or ethyl ester form.

17. The method of claim 11, wherein the enteral administration comprises oral administration.

18. The method of claim 11, wherein the enteral administration of the omega-3 fatty acid composition is concurrent with PN.

19. The method of claim 11, wherein preventing comprises an enhanced ability for the subject to tolerate enteral feeding as compared to a subject receiving PN but not administered an effective amount of an omega-3 fatty acid composition.

20. A method of advancing enteral tolerance in a subject receiving parenteral nutrition, the method comprising: providing a subject receiving parenteral nutrition; and administering to the subject an effective amount of an omega-3 fatty acid composition, wherein the omega-3 fatty acid composition is administered enterally.

21. The method of claim 20, wherein the subject is an infant having a low birth weight, very low birth weight, extremely low birth weight, a low gestational age, SBS, NEC, gastroeschesis, omphelacele, atresias, Hirschprungs disease, functional short bowel syndrome or a combination thereof.

22. The method of claim 20, wherein the subject is diagnosed with PNALD, wherein PNALD is defined as having a direct bilirubin concentration of >2 mg/dL and/or elevated transaminases, GGT, alk phos, or clinical correlation.

23. The method of claim 20, wherein the omega-3 fatty acid composition comprises DHA and EPA.

24. The method of claim 20, wherein the omega-3 fatty acid composition comprises fish oil or deodorized fish oil.

25. The method of claim 20, wherein the omega-3 fatty acid composition comprises algal sourced omega-3 fatty acids.

26. The method of claim 25, wherein the algal sourced omega-3 fatty acids are in the triglyceride or ethyl ester form.

27. The method of claim 20, wherein the enteral administration comprises oral administration.

28. The method of claim 20, wherein the enteral administration of the omega-3 fatty acid composition is concurrent with PN.

29. The method of claim 20, wherein advancing enteral tolerance comprises an enhanced ability for the subject to tolerate enteral feeding as compared to a subject receiving PN but not administered an effective amount of an omega-3 fatty acid composition.

30-43. (canceled)

44. A method of attenuating hepatocellular apoptosis in a subject receiving parenteral nutrition or suffering from PNALD, the method comprising: providing a subject receiving parenteral nutrition or a subject suffering from PNALD; and administering to the subject an effective amount of an omega-3 fatty acid composition, wherein the omega-3 fatty acid composition is administered enterally.

45. The method of claim 44, wherein the subject has levels of retained hydrophilic bile salts that are higher than the levels of retained hydrophilic bile salts in a subject not receiving parenteral nutrition or suffering from PNALD.

46. The method of claim 44, wherein the subject is an infant having a low birth weight, very low birth weight, extremely low birth weight, a low gestational age, short bowel syndrome (SBS), necrotizing entercolitis (NEC), gastroeschesis, omphelacele, atresias, Hirschprungs disease, functional short bowel syndrome or a combination thereof.

47. The method of claim 44, wherein a subject with PNALD is a subject having a direct bilirubin concentration of >2 mg/dL and/or elevated transaminases, GGT, alk phos, or clinical correlation.

48. The method of claim 44, wherein the omega-3 fatty acid composition comprises docosahexanoic acid (DHA) and eicosapentaenoic acid (EPA).

49. The method of claim 44, wherein the omega-3 fatty acid composition comprises fish oil or deodorized fish oil.

50. The method of claim 44, wherein the omega-3 fatty acid composition comprises algal sourced DHA and EPA.

51. The method of claim 44, wherein the enteral administration comprises oral administration.

52. The method of claim 44, wherein attenuating hepatocellular apoptosis comprises reducing the level of hepatocellular apoptosis to a level that is lower than the level of hepatocellular apoptosis in a subject receiving parenteral nutrition or suffering from PNALD but not receiving an effective amount of an omega-3 fatty acid composition.
